(19) United States
(12) Patent Application Publication
(10) Pub. No.: US 2008/0039499 A1
(43) Pub. Date: Feb. 14, 2008

(54) Chemical Compounds

(76) Inventors: Justin Fairfield Bower, Cheshire (GB); Gordon Alexander Hamlin, Cheshire (GB); Jon Winter, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 11/628,318

(22) PCT Filed: May 31, 2005
(86) PCT No.: PCT/GB05/02142

§ 371 Date: Dec. 1, 2006

(30) Foreign Application Priority Data
Jun. 4, 2004 (GB) 0412468.1

(51) Int. Cl. C07D 417/12; A61K 31/426; A61K 31/445; A61K 31/4535; C07D 417/14; A61K 31/454; A61P 29/00; C07D 277/38

(52) U.S. Cl. 514/321; 514/323; 514/326; 514/371; 546/209; 546/213; 548/195

(57) ABSTRACT

The use of a compound of formula (I) or a pharmaceutically acceptable salt or solvate thereof, wherein R¹, R^{a}, R⁸, R², R³ and R⁴ are as defined in the specification, in the preparation of a medicament for the treatment of C—C chemokine mediated conditions. Novel compounds of formula (I) and pharmaceutical compositions containing them are also described and claimed.

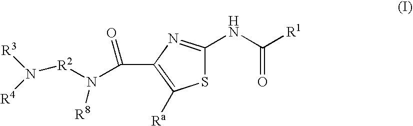

The present invention relates to pharmaceutical compositions which comprise compounds that act via antagonism of the CCR2b receptor for which MCP-1 is one of the known ligands, and so may be used to treat inflammatory disease which is mediated by these receptors. These compounds contain a cyclic aromatic moiety. The invention further relates to novel compounds for use in the compositions, to processes for their preparation, to intermediates useful in their preparation and to their use as therapeutic agents.

Chemokines play an important role in immune and inflammatory responses in various diseases and disorders, including rheumatoid arthritis, chronic obstructive pulmonary disease, atherosclerosis and other autoimmune pathologies such as inflammatory bowel disease, diabetes, asthma and allergic diseases. Chemokines also have a role in angiogenesis and modulation of chemokines may be beneficial in the treatment of cancer. Chemokines are small secreted molecules belonging to a growing superfamily of 8-14 kDa proteins characterized by a conserved four cysteine motif. The chemokine superfamily can be divided into two main groups exhibiting characteristic structural motifs, the Cys-X-Cys (C—X—C) and Cys-Cys (C—C) families. These are distinguished on the basis of a single amino acid insertion between the NH-proximal pair of cysteine residues and sequence similarity.

The C—C chemokines include potent chemoattractants of monocytes and lymphocytes such as monocyte chemoattractant proteins 1-3 (MCP-1, MCP-2 and MCP-3), RANTES (Regulated on activation, Normal T expressed and Secreted), eotaxin and the macrophage inflammatory proteins 1α and 1β (MIP-1α and MIP-1β).

The C—X—C chemokines include several potent chemoattractants and activators of neutrophils such as interleukin-8 (IL-8) and neutrophil-activating peptide 2 (NAP-2).

Studies have demonstrated that the actions of chemokines are mediated by subfamilies of G-protein coupled receptors, among which there are the receptors designated CCR1, CCR2, CCR2A, CCR2B, CCR3, CCR4, CCR5, CCR6, CCR7, CCR8, CCR9, CCR10, CXCR1, CXCR2, CXCR3, CXCR4, CXCR5 and CX3CR1. These receptors represent good targets for drug development since agents which modulate these receptors would be useful in the treatment of disorders and diseases such as those mentioned above.

U.S. Pat. No. 5,712,270 describes a range of thiazole derivatives useful for the treatment of neurological diseases.

The applicants have found a class of compounds containing a cyclic moiety which have useful antagonism of C—C chemokine receptors and in particular of the CCR2b receptor.

The present invention provides the use of a compound of formula (I) 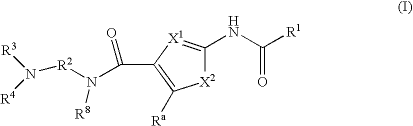
or a pharmaceutically acceptable salt or solvate thereof,
wherein
X¹ is nitrogen, or CH,
X² is sulphur or NH,
R¹ is an optionally substituted alkyl, optionally substituted alkoxy, optionally substituted cycloalkyl, optionally substituted heterocyclyl or optionally substituted aryl ring, wherein two substituents may be joined together to form an optionally substituted fused bicyclic ring, which may contain hetero atoms,
R^{a} is hydrogen, C₁₋₃alkyl, C₂₋₄alkenyl, C₂₋₄alkynyl, trifluoromethyl, halo, amino, C₁₋₃alkylamino, di-C₁₋₃alkylamino, C₁₋₄alkoxy, hydroxy, thioC₁₋₄alkyl, or cyclopropyl;
R⁸ is hydrogen or an optionally substituted C₁₋₄alkyl group,
R² is an optionally substituted C₂₋₁₀straight or branched alkylene group, which is optionally interposed with a group NR^{b} where R^{b} is hydrogen or a C₁₋₃methyl group; or
R² together with R⁸ and the nitrogen atoms to which they are attached may form an optionally substituted cycloalkyl or heterocyclic ring,
R³ and R⁴ are independently selected from an optionally substituted C₁₋₁₀ alkyl group, an optionally substituted C₂₋₁₀ alkenyl group, an optionally substituted C₁₋₁₀ alkynyl group or an optionally substituted heterocyclic group,
or R³ and R⁴ together with the nitrogen atom to which they are attached form an optionally substituted heterocyclic ring, which optionally contains additional heteroatoms,
or R³ together with R² or R⁸ and the nitrogen atom(s) to which they are attached form an optionally substituted heterocyclic ring which optionally contains additional heteroatoms,
or R³ and R⁴ together with R² form an optionally substituted bridged ring structure, in the preparation of a medicament for the treatment of C—C chemokine mediated conditions.

Compounds of formula (I) can be used in the treatment of diseases in which the chemokine receptor belongs to the C—C receptor subfamily, more preferably the target chemokine receptor is the CCR2 receptor.

CCR2 is a receptor for the Monocyte chemoattractant protein-1 (MCP-1). MCP-1 is a member of the chemokine family of pro-inflammatory proteins which mediate leukocyte chemotaxis and activation. MCP-1 is a C—C chemokine which is potent T-cell and monocyte chemoattractant. MCP-1 has been implicated in the pathophysiology of a large number of inflammatory diseases including rheumatoid arthritis, chronic obstructive pulmonary disease, atherosclerosis and inflammatory bowel disease.

MCP-1 acts through the CCR2 receptor. MCP-2, MCP-3 and MCP-4 may also act, at least in part, through this receptor. Therefore in this specification, when reference is made to “inhibition or antagonism of MCP-1” or “MCP-1 mediated effects” this includes inhibition or antagonism of MCP-2 and/or MCP-3 and/or MCP-4 mediated effects when MCP-2 and/or MCP-3 and/or MCP-4 are acting through the CCR2 receptor.

A compound of formula (I), or a pharmaceutically acceptable salt thereof, can be used in the treatment of:
(1) (respiratory tract)—obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus.
(2) (bone and joints) arthritides associated with or including osteoarthritis/osteoarthrosis, both primary and secondary to e.g. congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behçet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies.
(3) (skin) psoriasis, atopic dermatitis, contact dermatitis or other eczematous dermatoses, and delayed-type hypersensitivity reactions; phyto- and photo dermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions.
(4) (eyes) blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial.
(5) (gastrointestinal tract) glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema).
(6) (abdominal) hepatitis, including autoimmune, alcoholic and viral; fibrosis and cirrhosis of the liver; cholecystitis; pancreatitis, both acute and chronic.
(7) (genitourinary) nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female).
(8) (Allograft rejection) acute and chronic following, for example, transplantation of kidney, heart, liver, lung, bone marrow, skin or cornea or following blood transfusion; or chronic graft versus host disease;
(9) (CNS) Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes.
(10) Other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome.
(11) Other disorders with an inflammatory or immunological component; including acquired immune deficiency syndrome (AIDS), leprosy, Sezary syndrome, and paraneoplastic syndromes.
(12) Cardiovascular; atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (e.g. syphilitic); vasculitides; disorders of the proximal and peripheral veins including phlebitis and thrombosis, including deep vein thrombosis and complications of varicose veins.
(13) (Oncology) treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumor recurrences, and paraneoplastic syndromes. The applicants have found that the introduction of a tertiary amine in the side chain is particularly advantageous in compounds of this type.

As used herein, the term “heteroatom” refers to non-carbon atoms such as oxygen, nitrogen or sulphur atoms. The term ‘alkyl’ when used either alone or as a suffix includes straight chain and branched structures. These groups may contain up to 10, preferably up to 6 and more preferably up to 4 carbon atoms. Similarly the terms “alkenyl” and “alkynyl” refer to unsaturated straight or branched structures containing for example from 2 to 10, preferably from 2 to 6 carbon atoms. Cyclic moieties such as cycloalkyl, cycloalkenyl and cycloalkynyl are similar in nature but have at least 3 carbon atoms. They may be bridged. Terms such as “alkoxy” and “alkanoyl” comprise alkyl moieties as defined above, attached to the appropriate functionality.

The term “halo” includes fluoro, chloro, bromo and iodo. References to aryl groups include aromatic carbocylic groups such as phenyl and naphthyl. The term “heterocyclyl” includes aromatic or non-aromatic rings, or partially unsaturated ring systems, for example containing from 4 to 20, suitably from 5 to 10 ring atoms, at least one of which is a heteroatom such as oxygen, sulphur or nitrogen. Rings may be mono-, bi- or tricyclic. They may also contain bridges, in particular alkyl bridges. Examples of such groups include furyl, thienyl, pyrrolyl, pyrrolidinyl, imidazolyl, thiazolyl, tetrazolyl, oxazolyl, isoxazolyl, pyrazolyl, pyridyl, pyrimidinyl, pyrazinyl, pyridazinyl, triazinyl, quinolinyl, iosquinolinyl, quinoxalinyl, benzthiazolyl, benzoxazolyl, benzothienyl, benzofuranyl, tetrahydrofuryl, chromanyl, benzothienyl, piperidinyl, 1,2,3,4-tetrahydroquinolinyl, 1,2,3,4-tetrahydroisoquinolinyl, pyrazinyl, piperazinyl, pyrimidinyl, pyridazinyl, quinoxalinyl, quinazolinyl, cinnolinyl, indolyl, indolinyl, benzimidazolyl, pyrazolyl, indazolyl, oxazolyl, benzoxazolyl, isoxazolyl, isothiazolyl, morpholinyl, dioxolane, benzodioxolane, 4H-1,4-benzoxazinyl, 4H-1,4-benzothiazinyl, 1,2,3-triazolyl, 1,2,4-triazolyl, oxadiazolyl, furazanyl, thiadiazolyl, dibenzofuranyl, dibenzothienyl oxiranyl, oxetanyl, azetidinyl, piperidinyl, oxepanyl, oxazepanyl, tetrahydro-1,4-thiazinyl, 1,1-dioxotetrahydro-1,4-thiazinyl, homopiperidinyl, homopiperazinyl, dihydropyridinyl, tetrahydropyridinyl, dihydropyrimidinyl, tetrahydropyrimidinyl, tetrahydrothienyl, tetrahydrothiopyranyl or thiomorpholinyl.

“Heteroaryl” refers to those groups described above which have an aromatic character. The term “aralkyl” refers to aryl substituted alkyl groups such as benzyl.

Other expressions used in the specification include “hydrocarbyl” which refers to any structure comprising carbon and hydrogen atoms. For example, these may be alkyl, alkenyl, alkynyl, aryl, aralkyl, cycloalkyl, cycloalkenyl or cycloalkynyl.

In a particular embodiment, X¹ is N.

X² is suitably S.

In one embodiment of the invention, R¹ is optionally substituted aryl, and in particular optionally substituted phenyl or naphthyl. Suitably, R¹ is substituted phenyl.

Where R¹ is optionally substituted cycloalkyl, it is suitably an optionally substituted C₅₋₇cycloalkyl group, such as cyclohexyl.

Suitable heterocyclic groups for R¹ include heteroaryl groups an in particular pyridyl.

Suitable alkyl groups R¹ are branched C₃₋₁₀alkyl groups such as tert-butyl. Similarly alkoxy groups R¹ will suitably contain such alkyl groups, and a particular example of R¹ is tert-butyloxy.

Suitable optional substituents for alkyl, alkoxy, cycloalkyl, aryl groups or heterocyclic groups R¹ include from 1 to 4, suitably from 1 to 3 groups selected from functional groups, hydrocarbyl groups such as alkyl groups, alkenyl, alkynyl groups or aralkyl groups, or heterocyclic groups.

As used herein, the term “functional group” refers to reactive substituents. They may comprise electron-donating or electron-withdrawing groups. Examples of such groups include halo, oxo, cyano, nitro, C(O)ₙR¹¹, OR¹¹, S(O)_{q}R¹¹, NR¹²R¹³, C(O)NR¹²R¹³, OC(O)NR¹²R¹³, —CH═NOR¹¹, —NR¹²C(O)ₙR¹¹, —NR¹¹CONR¹²R¹³, —N═CR¹²R¹³, S(O)_{q}NR¹²R¹³ or —NR¹²S(O)_{q}R¹¹ where R¹¹, R¹² and R¹³ are independently selected from hydrogen, optionally substituted hydrocarbyl or optionally substituted heterocyclyl, or R¹² and R¹³ together form an optionally substituted ring which optionally contains further heteroatoms such as S(O)_{q′}, oxygen and nitrogen, n is an integer of 1 or 2, q is 0 or an integer selected from 1, 2 or 3, and q′ is 0, 1 or 2. Where functional groups comprise S(O)_{q}NR¹²R¹³ or —NR¹²S(O)_{q}R¹¹, q is generally an integer of 1, 2 or 3, and suitably 1 or 2.

Suitable optional substituents for hydrocarbyl or heterocyclic groups R¹¹, R¹² and R¹³ include halo, (including perhaloalkyl such as trifluoromethyl), mercapto, hydroxy, alkoxy, oxo, heteroaryloxy, alkenyloxy, alkynyloxy, alkoxyalkoxy, aryloxy (where the aryl group may be substituted by halo, nitro, or hydroxy), cyano, nitro, amino, mono- or di-alkyl amino, alkylamido, oximino (for example hydroxyimino or alkyloxyimino) or S(O)_{q}R^{y} where q is as defined above and R^{y} is alkyl.

Particular substituents for R¹ include one or more groups selected from alkyl groups, in particular C₁₋₄alkyl groups such as methyl, C₂₋₄ alkenyl, or alkynyl groups such as ethynyl, benzyl, a saturated heterocyclic group such as tetrahydropyranyl, or a functional group as defined above. Particular functional groups which can form substituents on R¹ include halo, cyano, C(O)ₙ¹¹, OR¹¹ and S(O)_{q}R¹¹. Particular examples of R¹¹ are hydrogen, alkyl, or aryl, and in particular methyl or phenyl.

A particular example of n is 1. A particular example of q is 0.

Thus examples of substituents for R¹ are one or more halo groups (such as chloro or fluoro), hydroxy, methoxy, cyano, methyl, methylthio, acetyl, ethynyl, benzyl or phenylsulphonyl.

Examples of R¹ groups include phenyl, 2-pyridyl, 3-pyridyl, 4-pyridyl or a group (a)-(u)

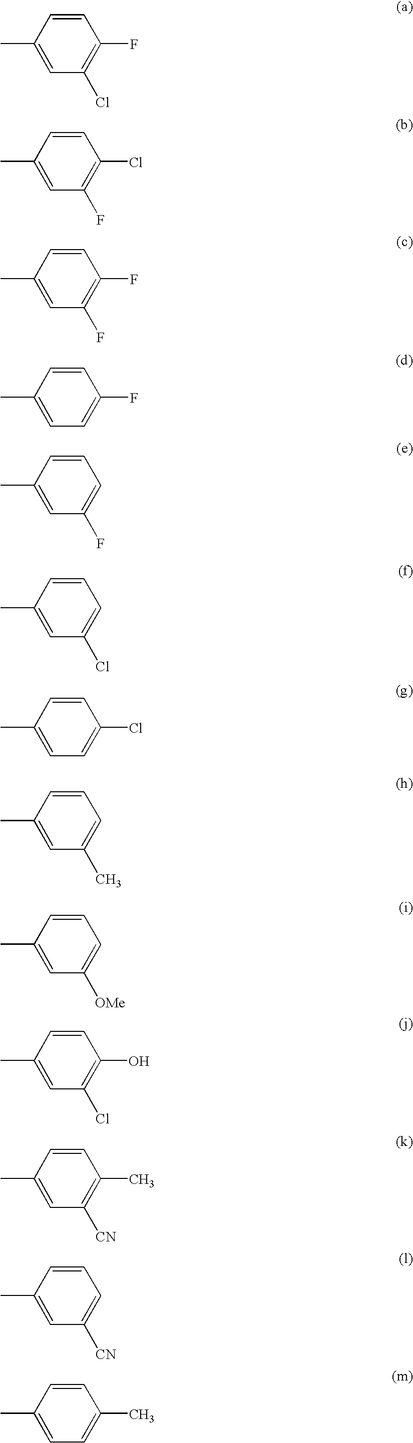

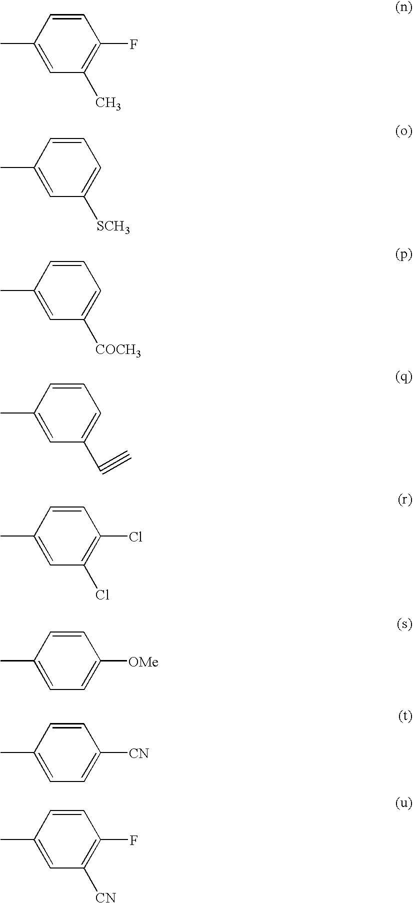

Suitably R¹ is substituted by one or two halo groups, which are preferably selected from chloro or fluoro.

A specific example of an R¹ group is 2-chloro-3-fluorophenyl.

Alternatively, two substituents on R¹ may be linked together to form an optionally substituted fused bicyclic ring system. Preferably the fused bicyclic ring is of formula (i)

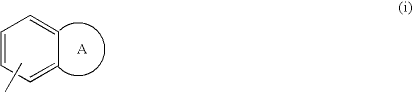

where A is an optionally substituted 4-7 membered ring which may contain one or more heteroatoms. Any substituents on R¹ as described above, may be located on the ring A of the R¹ group. Particularly suitable optional substituents for the ring A include functional groups, heterocyclic groups or hydrocarbyl groups such as alkyl or aralkyl groups. The ring A may be saturated or unsaturated. When unsaturated, it may be aromatic in character. Suitably ring A forms a fused five or six membered ring.

Preferably ring A includes at least one heteroatom.

Particular examples of bicyclic groups R¹ include groups of sub-formulae (v)-(f′)

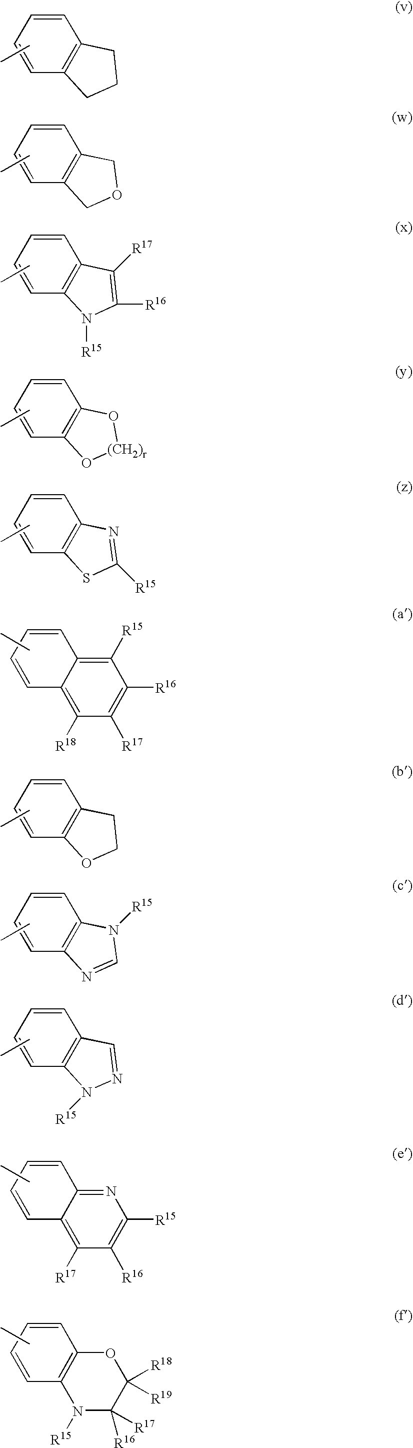

where r is 1, 2 or 3, and R¹⁵, R¹⁶, R^{17,} R¹⁸ and R¹⁹ are independently selected from hydrogen or R¹ substituents as described above. In particular, where R¹⁵, R¹⁶, R^{17,} R¹⁸ and R²⁰ are other than hydrogen, they are selected from alkyl such as methyl, methoxy, benzyl, piperidinyl, or phenylsulphonyl, or where two of R¹⁶, R¹⁷, R¹⁸ and R¹⁹ are on the same carbon atom, they may form an oxo substituent. Particular examples of such groups are illustrated hereinafter.

R^{a} is suitably hydrogen or a small substituent such as methyl, trifluoromethyl or amino, and preferably R^{a} is hydrogen.

Where R⁸ is an optionally substituted alkyl group, suitable optional substituents include functional groups as defined above. Preferably R⁸ is unsubstituted.

Suitably, R² is an optionally substituted C₂₋₆straight or branched alkylene group, in particular, a C₂₋₃alkylene group. Preferably R² is unsubstituted. Where it is substituted, suitable substituents include functional groups as defined above.

Where R² is an alkylene chain which is interposed by a group NR^{b}, this group will not be at the end position of the chain. Preferably R^{b} is hydrogen.

In particular R² and R⁸ may together with the nitrogen atom to which they are attached form a heterocyclic ring, in particular, a saturated heterocyclic ring such as piperidine.

Where R³ or R⁴ comprises an optionally substituted C₁₋₁₀ alkyl group, an optionally substituted C₂₋₁₀ alkenyl group, an optionally substituted C₂₋₁₀ alkynyl group or an optionally substituted heterocyclic group, suitable optional substituents include functional groups, such as cyano, oxo, carboxy, cycloalkyl groups, aryl groups or heterocyclic groups where any cycloalkyl, aryl or heterocyclic substituents may themselves be optionally substituted by one or more functional groups, optionally substituted hydrocarbyl groups such as optionally substituted alkyl, or heterocyclic groups.

In a particular embodiment R³ or R⁴ are optionally substituted C₁₋₁₀alkyl groups.

Suitably R³ and/or R⁴ is methyl, ethyl, n-propyl, n-butyl, n-pentyl or n-hexyl, and in particular methyl or ethyl.

When C₁₋₁₀alkyl groups R³ or R⁴ have a substituent which is a functional group, particular examples include cyano, C(O)ₙR¹¹ such as carboxy or methyl carboxylate, OR¹¹ such as hydroxy or methoxy, or S(O)_{q}R¹¹ such as thioC₁₋₃alkyl, for instance thiomethyl, or methylsulphonyl where n, q and R¹¹ are as defined above. In particular R¹¹ in this instance is selected from heterocyclic such as morpholino, or aryl such as phenyl.

In particular, where R³ or R⁴ are alkyl groups, they are optionally substituted by a heterocyclic group which may itself be optionally substituted. Particular examples of heterocyclic groups include furyl, tetrahydrofuryl thienyl, pyrrolyl, oxazolyl, isoxazolyl, thiazolyl, thiadiazolyl, isothiazolyl, imidazolyl, pyrazolyl, pyrrolidinyl, imidazolyl, pyridyl, pyrimidinyl, oxanyl, indolyl, quinolyl, isoquinolyl, piperidinyl, piperazinyl, dioxolanyl, benzo-1,3-dioxolyl, 2,3-dihydroindole, or thiiranyl.

In addition, R³ or R⁴ may comprise an alkyl group that is optionally substituted by an aryl such as phenyl, or cycloalkyl group such as cyclopropyl group, either of which may themselves be optionally substituted.

Where these aryl, cycloalkyl or heterocyclic substituents on R³ and R⁴ are themselves substituted, those substituents are suitably selected from C₁₋₃alkyl groups which optionally carry such a functional group as a substituent, or functional groups as defined above. Particular functional groups in this case include halo such as fluoro, cyano, oxo (where the ring is at least partially unsaturated) C(O)ₙR¹¹ such as carboxy or methyl carboxylate, OR¹¹ such as hydroxy or methoxy, or S(O)_{q}R¹¹ such as thioC₁₋₃alkyl, for instance thiomethyl, or methylsulphonyl where n, q and R¹¹ are as defined above,

Preferably, when R³ or R⁴ is an alkyl group, it is substituted as described above.

In an alternative embodiment, R³ and R⁴ together with the nitrogen atom to which they are attached form an optionally substituted heterocyclic ring, which optionally contains additional heteroatoms. In particular, these rings are saturated rings. Examples of these are compounds of formula (I) where formula R⁴R³N— comprise a group of sub-formula (xx)-(xxv).

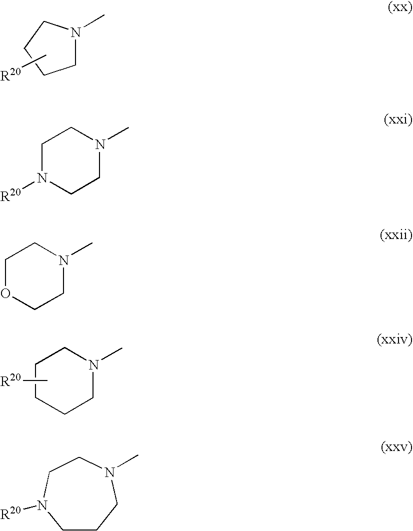

where R²⁰ is hydrogen or a substituent.

In particular the group of formula R⁴R³N— is a group (xxi) or (xxiv).

Suitable substituents R²⁰ include alkyl, in particular C₁₋₄alkyl such as methyl, aralkyl such as benzyl, optionally substituted heterocyclic groups, in particular saturated heterocyclic groups such as pyrrolidinyl or piperidinyl which may themselves be optionally substituted, and functional groups such as cyano, —NR¹²R¹³, C(O)ₙR¹¹, OR¹¹, or S(O)_{q}R¹¹ where n, q, R¹¹ R¹² and R¹³ are as defined above. Particular functional groups C(O)ₙR¹¹ include carboxy or methyl carboxylate. Particular functional groups OR¹¹ are hydroxy or methoxy. Particular functional groups S(O)_{q}R¹¹ are thioC₁₋₃alkyl, for instance thiomethyl, or methylsulphonyl, as well as phenylsulphonyl.

When R²⁰ is a heterocyclic group, it may be optionally substituted by a functional group, in particular a functional group as listed above for R²⁰.

In yet another embodiment, R³ together with R² or R⁸ and the nitrogen atom(s) to which they are attached form an optionally substituted heterocyclic ring which optionally contains additional heteroatoms. Where R³ together with R² together with the nitrogen atom to which they are attached forms a ring, the attachment may take place at any suitable carbon atom within the R² chain, but is suitably at the R² carbon that is directly adjacent to the group Y. Thus, suitable examples of the group of sub-formula (x) 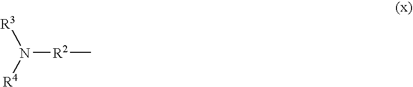
include groups of sub-formula (bb) or (cc) 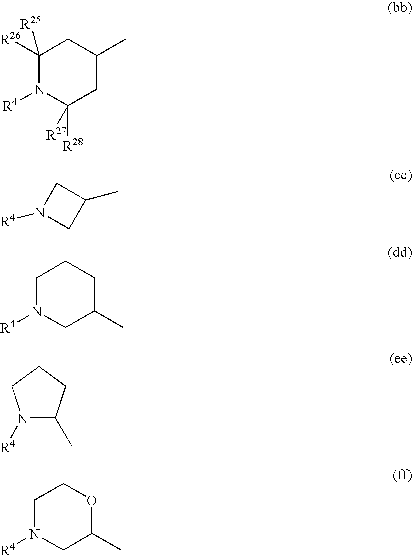
where R⁴ is as defined above, and R²⁵, R²⁶, R²⁷ and R²⁸ are independently selected from hydrogen or C₁₋₃alkyl such as methyl. Preferably R²⁵, R²⁶, R²⁷ and R²⁸ are all hydrogen, or all methyl, and most preferably, they are all hydrogen.

A particularly preferred group of (x) is a group of formula (bb) above.

Thus is a particular embodiment, the invention provides the use as described above of compound of formula IB 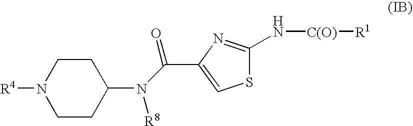
where R¹, R⁴ and R⁸ are as defined above.

Particular examples of groups R⁴ in the groups of sub-formula (bb) to (ff) include those listed in Table 1.

TABLE 1

| Designation | R⁴ |
|---|---|
| 1a | —(CH₂)₂CH₃ |
| 1b | —(CH₂)₂OCH₃ |
| 1c | —CH₃ |
| 1d | 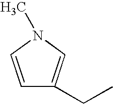 |
| 1e | 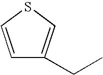 |
| 1f | 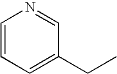 |
| 1g | 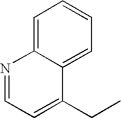 |
| 1h | —(CH₂)₃CH₃ |
| 1i | —CH₂CH═C(CH₃)₂ |
| 1j | 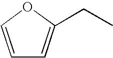 |
| 1k | 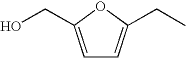 |
| 1l | 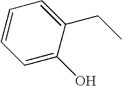 |
| 1m | 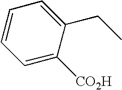 |
| 1n | 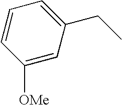 |
| 1o | 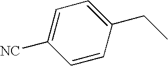 |
| 1p | 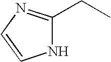 |
| 1q | 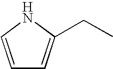 |
| 1r | 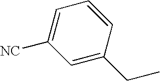 |
| 1s | 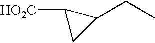 |
| 1t | 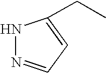 |
| 1u | 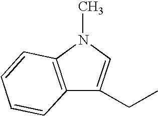 |
| 1v | 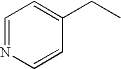 |
| 1w | 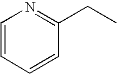 |
| 1x | 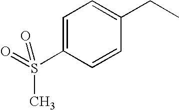 |
| 1y | 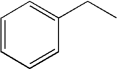 |
| 1z | 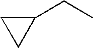 |
| 2a | 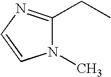 |
| 2b | 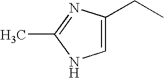 |
| 2c | 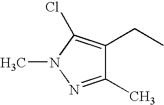 |
| 2d | 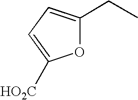 |
| 2e | —(CH₂)₂OH |
| 2f | 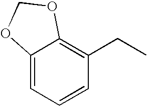 |
| 2g | 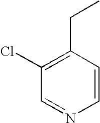 |
| 2h | —(CH₂)₅CO₂CH₃ |
| 2i | 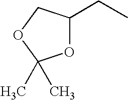 |
| 2j | 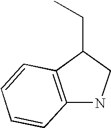 |
| 2k | 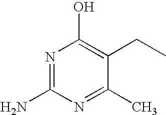 |
| 2l | 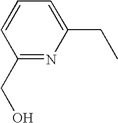 |
| 2m | 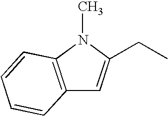 |
| 2n | 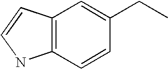 |
| 2o | 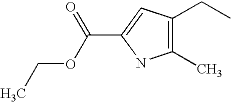 |
| 2p | 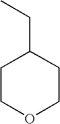 |
| 2q | 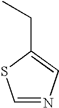 |
| 2r | 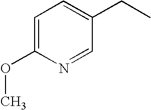 |
| 2s | 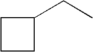 |
| 2t | 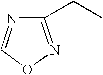 |
| 2u | 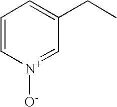 |
| 2v | 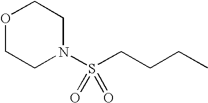 |
| 2w | 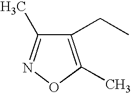 |
| 2x | 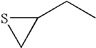 |
| 2y | 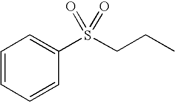 |
| 2z | 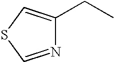 |
| 3a | —CH(CH₃)C≡CH |
| 3b | 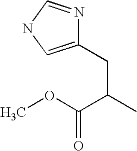 |
| 3c | —CH(CH₃)C(O)N(CH₂CH₃)₂ |
| 3d | 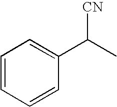 |
| 3e | —CH₂OCH₃ |
| 3f | 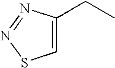 |
| 3g | —CH₂CH₃ |
| 3h | 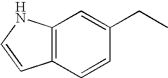 |
| 3i | 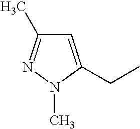 |
| 3j | —CH(CH₃)₂ |
| 3k | —CH(CH₃)C(O)CH₃ |
| 3l | 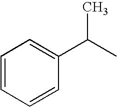 |
| 3m | 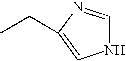 |
| 3n | 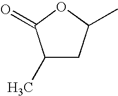 |
| 3o | 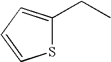 |
| 3p | 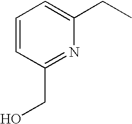 |
| 3q | 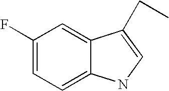 |
| 3r | 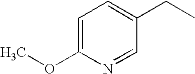 |
| 3s | —N(CH₃)₂ |
| 3t | 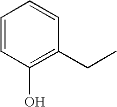 |
| 3u | 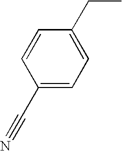 |
| 3v | 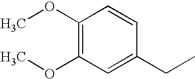 |
| 3w | —(CH₂)₂CH(CH₃)₂ |
| 3x | 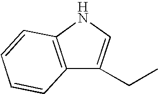 |
| 3y | 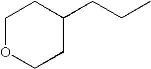 |
| 3z | 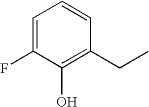 |
| 4a | 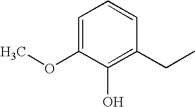 |
| 4b | 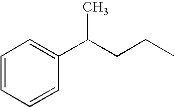 |
| 4c | 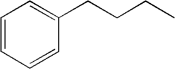 |
| 4d | 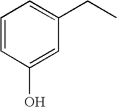 |
| 4e | 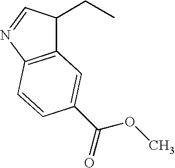 |
| 4f | 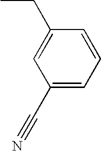 |
| 4g | 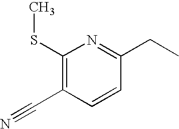 |
| 4h | 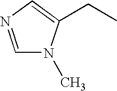 |
| 4i | 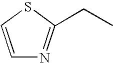 |
| 4j | 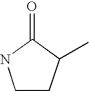 |
| 4k | 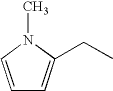 |

Particular examples of group R⁴ are groups of sub-formulae 1f, 1k, 1l, 1p, 1q, 1t, 1x, 1y, 1z, 2e, 2j, 2l, 2p, 3m, 3x and 4k above.

In a particularly preferred embodiment, the group R⁴ comprises alkyl substituted with heterocyclic group, which is itself optionally substituted as described above.

In another preferred embodiment, R⁴ is an alkyl group substituted with a substituted aryl group such as a substituted phenyl, wherein the substituents are as described above.

In yet another preferred embodiment, R⁴ is a group S(O)_{q}R¹¹ where q and R¹¹ are as defined above.

Where R³ together with R⁸ and the nitrogen atoms to which they are attached form an optionally substituted heterocyclic ring which contains additional heteratoms, suitable examples of the group of sub-formula (y) 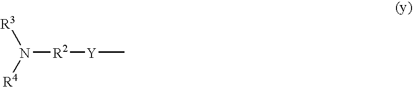
include groups of sub-formulae (g′) 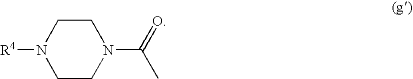
A particular example of such a group is 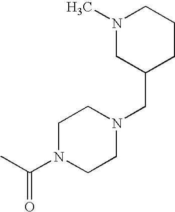
When R³ and R⁴ together with R² form an optionally substituted bridged ring structure, a particular structure is of formula (h′) 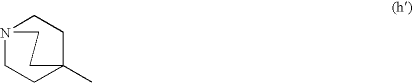
Suitable optional substituents are those described above for alkyl groups R³ or R⁴.

Many of the compounds of formula (I) are novel and these form a further aspect of the invention. Specifically, the invention further provides a compound of formula (IA) 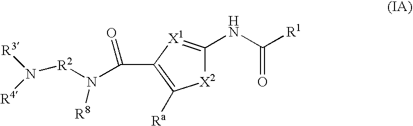
or a pharmaceutically acceptable salt or solvate thereof,
wherein X¹, X², R², R⁸, R^{a} and R¹ are as defined in relation to formula (I), and where R^{3′} and R^{4′} are equivalent to R³ and R⁴ as defined in relation to formula (I) respectively, provided that when R¹ is optionally substituted phenyl, and R^{a} and R⁸ is hydrogen, R^{3′} and R^{4′} are not both unsubstituted alkyl, or do not together with the nitrogen atom to which they are attached form a substituted piperazinyl ring, and
further provided that when R¹ is an optionally substituted phenyl, R^{a} is hydrogen, C₁₋₃alkyl, or halo; R⁸ is hydrogen or a C₁₋₄alkyl group,
and either R^{3′} and R² together with the nitrogen atom to which they are attached form a piperidinyl ring, or R^{3′} together R⁸ and the nitrogen atom(s) to which they are attached forms a piperazinyl ring, then R^{4′} is other than an unsubstituted C₁₋₆alkyl group, and yet further provided that when R¹ is an optionally substituted phenyl, R^{a} is hydrogen, C₁₋₃alkyl, or halo; and R² and R⁸ together with the nitrogen atom to which they are attached form a piperidinyl group, then R³′ and R^{4′} are not both unsubstituted C₁₋₆alkyl groups.

Preferred variables for the groups X¹, X², R¹ R², R^{a}, R⁸, R² are as defined above. Preferred variables for R^{3′} and R^{4′} are as defined above for R³ and R⁴ respectively.

In particular, when any R^{4′} group is an alkyl group, it carries a substituent as described above.

Suitable pharmaceutically acceptable salts of compounds of formula (I) or (IA) include are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. In another aspect, where the compound is sufficiently basic, suitable salts include acid addition salts such as methanesulfonate, fumarate, hydrochloride, hydrobromide, citrate, maleate and salts formed with phosphoric and sulphuric acid. There may be more than one cation or anion depending on the number of charged functions and the valency of the cations or anions. A preferred pharmaceutically acceptable salt is a sodium salt.

Particular compounds of formula (I) and (IA) are listed below in Tables 2 and 3.

TABLE 2

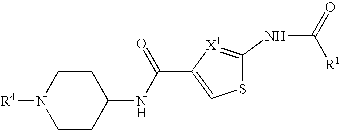

| | | | | LCMS | |
|---|---|---|---|---|---|
| Compd. | | | | M/z(+) | Chemistry |
| No. | X¹ | R⁴ | R¹ | (MH⁺) | Route |
| 1 | N | benzyl | 4-fluoro-3- | 473 | A or C |
| | | | chlorophenyl | | |
| 2 | N | 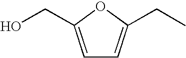 | 4-fluoro-3- chlorophenyl | 493 | D |
| 3 | N | 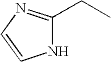 | 4-fluoro-3- chlorophenyl | 463 | D |
| 4 | N | 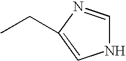 | 4-fluoro-3- chlorophenyl | 463 | D |
| 5 | N | benzyl | 3,4- | 457 | C |
| | | | difluorophenyl | | |
| 6 | N | 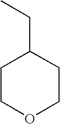 | 4-fluoro-3- chlorophenyl | 481 | D |
| 7 | N | —CH₂CH₂OH | 4-fluoro-3- | 427 | D |
| | | | chlorophenyl | | |
| 8 | N | 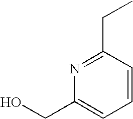 | 4-fluoro-3- chlorophenyl | 504 | D |
| 9 | N | 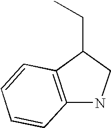 | 4-fluoro-3- chlorophenyl | 514 | D |
| 10 | N | 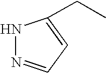 | 4-fluoro-3- chlorophenyl | 463 | D |
| 11 | N | 2-hydroxybenzyl | 4-fluoro-3- | 512 | D |
| | | | chlorophenyl | (M + Na⁺) | |
| 12 | N | 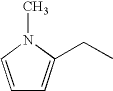 | 4-fluoro-3- chlorophenyl | 476 | D |
| 13 | N | 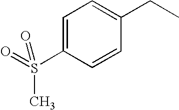 | 4-fluoro-3- chlorophenyl | 551 | D |
| 14 | N | 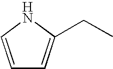 | 4-fluoro-3- chlorophenyl | 462 | D |
| 15 | N | 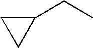 | 4-fluoro-3- chlorophenyl | 437 | D |
| 16 | N | 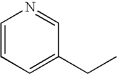 | 4-fluoro-3- chlorophenyl | 474 | D |
| 17 | N | benzyl | 3-chlorophenyl | 455 | B |
| 18 | N | benzyl | 4-pyridyl | 422 | B |
| 19 | N | benzyl | 2-pyridyl | 422 | B |
| 20 | N | benzyl | 3-pyridyl | 422 | B |
| 21 | N | benzyl | 3-fluorophenyl | 439 | B |
| 22 | N | benzyl | 3,4- | 489 | B |
| | | | dichlorophenyl | | |
| 23 | N | benzyl | 3-cyanophenyl | 446 | B |
| 24 | N | 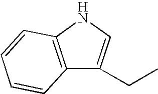 | 4-fluoro-3- chlorophenyl | 512 | D |
| 25 | N | benzyl | tert-butyloxy | 316 | C but stop |
| | | | | (MH⁺ | before Boc |
| | | | | minus t- | deprotection |
| | | | | butyl) | |
| 26 | CH | benzyl | 4-fluoro-3- | 472/ | E |
| | | | chlorophenyl | 474 | |
| 27 | N | 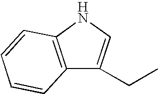 | 4-fluorophenyl | 478 | F |
| 28 | N | 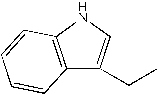 | 2-naphthyl | 510 | F |
| 29 | N | 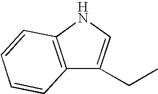 | 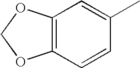 | 504 | F |
| 30 | N | 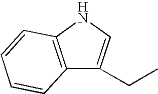 | 3-thienyl | 466 | F |

TABLE 3

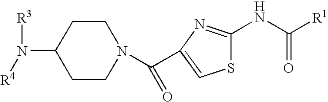

| | | | LCMS | |
|---|---|---|---|---|
| Compound | | | M/z(+) | Chemistry |
| No | R⁴R³N— | R¹ | (MH⁺) | Route |
| 27 | 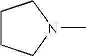 | 4-fluoro-3- chlorophenyl | 437 | A |

The chemical routes used to synthesize the examples and certain intermediates in their preparation are designated A-F and illustrated hereinafter.

Compounds of formula (I) and (IA) are suitably prepared by various routes which would be apparent to a chemist, and routes to compounds of formula (IA) form a further aspect of the invention. In particular compounds of formula (I) may be obtained by reacting a compound of formula (IV) 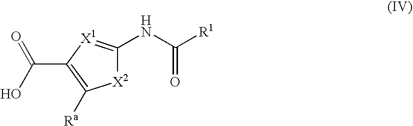
where X¹, X², R¹ and R^{a} are as defined in relation to formula (I), with a compound of formula (V) 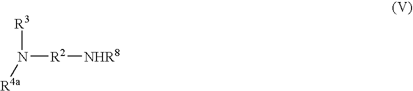
where R², R³ and R⁸ are as defined in relation to formula (I) and R^{4a} is a group R⁴ as defined in relation to formula (I), or a precursor thereof; and thereafter, if desired or necessary, converting any precursor groups R^{4a} to a group R⁴.

The reaction is suitably effected in an organic solvent such as dimethylformamide, in the presence of a base such as N,N-diisopropylethylamine and HATU at ambient temperature.

Examples of precursor groups R^{4a} include amine protecting groups such as tertiary butyloxycarbonyl (Boc) groups, which may be removed using conventional deprotection methods. Thereafter, the hydrogen group may be replaced by an alternative R⁴ group by an alkylation reaction or reductive animation reaction.

For instance, for the preparation of compounds of formula (I) where R³ and R² together with the nitrogen to which they are attached form a heterocyclic ring, for instance so that the group of formula (x) above is a group of formula (bb)-(ff), they may be prepared by reacting a compound of formula (VI) 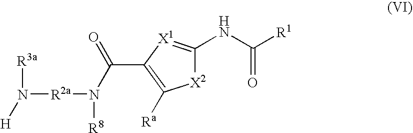
where X¹, X², R¹, R^{a} and R⁸ are as defined in relation to formula (I), R^{3a} and R^{2a} together with the nitrogen atom to which they are attached form a ring, with a compound of formula (X)
R⁴-R⁵¹ (VII)
where R⁴ is as defined in relation to formula (I), and R⁵¹ is a leaving group, such as halo, and in particular bromo. The reaction is suitably carried out in an organic solvent such as dimethylformamide, in the presence of a base such as an alkali metal carbonate, for instance potassium carbonate. Moderate temperatures for example of from 0 to 50° C., and conveniently at ambient temperature, are suitably employed.

Alternatively, where R⁴ is an optionally substituted alkyl group, the compound of formula (VI) may be reacted with a compound of formula (VIII)
R^{4x}—C(O)H (VIII)
where a group R^{4x}—CH₂— is equivalent to the desired R⁴ group, in the presence of a mild reducing agent. This reaction is suitably effected in an organic solvent such as tetrahydrofuran at moderate temperatures for example of from 0 to 50° C., and conveniently at ambient temperature. A suitable reducing agent is sodium triacetoxyborohydride.

In this case, the compounds of formula (VI) used is suitably in the form of a salt such as an acid addition salt, for example a trifluoroacetic acid salt.

Compounds of formula (VI) are suitably prepared by deprotecting a compound of formula (IX) 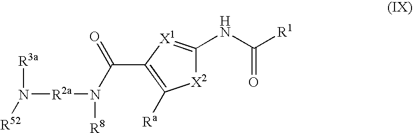
where X¹, X², R¹, R^{a} and R⁸ are as defined in relation to formula (I), R^{2a} and R^{3a} are as defined in relation to formula (VI) and R⁵² is an amine protecting group such as tertiary butyloxycarbonyl (Boc). Suitable deprotection conditions would be apparent to a skilled person, but may include treatment with an acid such as hydrochloric acid.

Compounds of formula (IV) above are suitably prepared by hydrolysis of a compound of formula (X) 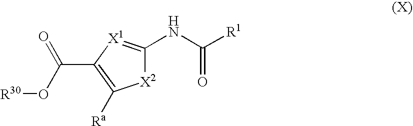
where X¹, X², R¹ and R^{a} are as defined in relation to formula (I), and R³⁰ is a hydrocarbyl group such as C₁₋₆alkyl.

Suitably hydrolysis is conducted in an organic solvent such as methanol-water, at temperatures such as 25 to 45° C. and using lithium hydroxide to effect hydrolysis.

Compounds of formula (X) are suitably prepared by reacting a compound of formula (XI) 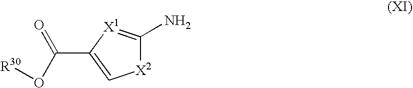
where X¹ and X² are as defined in relation to formula (I), R³⁰ is as defined in relation to formula (X), with a compound of formula (XII) 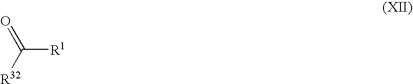
where R¹ is as defined in relation to formula (I) and R³² a leaving group such as halo.

The reaction is suitably effected in a solvent such as acetonitrile, dimethylsulphoxide (DMSO) or water, in the presence of a base such as diisopropylethylamine. Moderate temperatures, for example from 0 to 50° C. and conveniently, ambient temperatures are suitably employed.

Compounds of formula (I) may also be prepared by reacting a compound of formula (XIII) 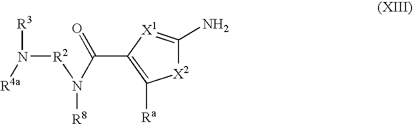
where X¹, X², R^{a}, R² R³ and R⁸ are as defined in relation to formula (I), R^{4a} is as defined in relation to formula (V), with a compound of formula (XIV) 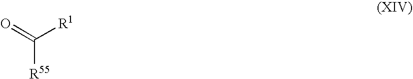
where R¹ are as defined in relation to formula (I) and R⁵⁵ is a leaving group such as halo, and in particular chloro, and thereafter if desired or necessary, converting any precursor groups R^{4a} to a group R⁴, for instance using the methods described above.

The reaction is suitably effected in a solvent such as acetonitrile, dimethylsulphoxide (DMSO) or water, in the presence of a base such as diisopropylethylamine. Moderate temperatures, for example from 0 to 50° C. and conveniently, ambient temperatures are suitably employed.

Compounds of formula (XIII) may be prepared by deprotection of a compound of formula (XV) 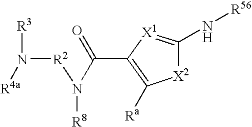
where X¹, X², R², R³, R⁸ and R^{a} are as defined in relation to formula (I), R^{4a} is as defined in relation to formula (V) and R⁵⁶ is a nitrogen protecting group, such as tertiary-butyloxycarbonyl (Boc). Conditions suitable for the removal of the protecting group would be apparent to a chemist, but may include acidification for example using an organic acid such as trifluoroacetic acid at elevated temperatures, for instance of from 50-90° C., and in particular at about 70° C.

Compounds of formula (XV) may be prepared by methods analogous to those described above in relation to the preparation of compounds of formula (I). For example, compounds of formula (XVI) 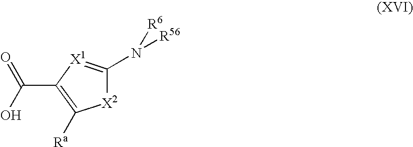
where R^{a}, and R⁵⁶ are as defined above, may be reacted with compounds of formula (V) as described above, using analogous conditions to those described for the reaction between compound (IV) and compound (V).

Alternatively, compounds of formula (XIII), in particular where X¹ is CH and X² is S may be prepared by reduction of a compound of formula (XVII) 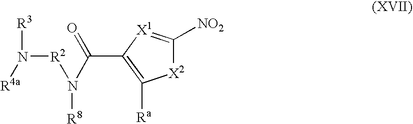
where X¹, X², R^{a}, R² R³ and R⁸ are as defined in relation to formula (I), R^{4a} is as defined in relation to formula (V). Reduction is suitably effected in the presence of a catalyst such as iron, and ammonium chloride, in an organic solvent such as ethanol/water. Elevated temperatures, for example from 50-100° C., and conveniently at the reflux temperature of the solvent are suitably employed.

Compounds of formula (XVII) are suitably prepared by reacting a compound of formula (XVIII) 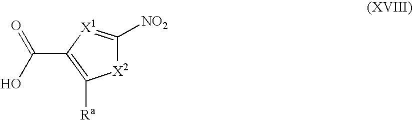
where X¹, X² and R^{a} are as defined in relation to formula (I), with a compound of formula (XIX) 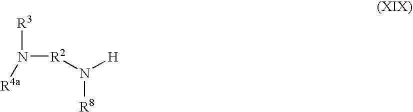
where R², R³ and R⁸ are as defined in relation to formula (I), R^{4a} is as defined in relation to formula (V). The reaction is suitably conducted in an organic solvent such as dichloromethane, in the presence of a base such as diisopropylethylamine, at moderate temperatures, such as ambient temperature.

Compounds of formulae (V), (VII), (VIII), (XI), (XII), (XIV), (XVIII) and (XIX) are either known compounds or they can be prepared from known compounds by conventional methods which would be readily apparent to a skilled chemist.

Variants of these processes may also be envisaged.

Novel intermediate compounds as defined above form a further aspect of the invention.

The invention further provides a compound of formula (I) or (IA) as defined above for use in the treatment of C—C-mediated disease such as inflammatory disease. When used in this way, the compounds are suitably formulated into pharmaceutical compositions which further contain a pharmaceutically acceptable carrier and these form a further aspect of the invention.

Furthermore, the invention provides the use of a compound of formula (I) as defined above in the preparation of a medicament for treating C—C chemokine mediated disease, and in particular for the treatment of CCR2B mediated inflammatory disease.

Some compounds of formula (I) and (IA) may possess chiral centres. It is to be understood that the invention encompasses the use of all such optical isomers and diasteroisomers as well as compounds of formula (IA) in any of these forms, and pharmaceutical compositions containing compounds of formula (IA).

The invention further relates to all tautomeric forms of the compounds of formula (IA) and pharmaceutical compositions containing these.

It is also to be understood that certain compounds of the formula (IA) can exist in solvated as well as unsolvated forms such as, for example, hydrated forms. It is to be understood that the invention encompasses all such solvated forms and pharmaceutical compositions containing these.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound of the Formula I will naturally vary according to the nature and severity of the conditions, the age and sex of the animal or patient and the route of administration, according to well known principles of medicine.

In using a compound of the Formula I for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

In a further aspect, the invention provides a method of treating inflammatory disease by administering a compound of formula (I) as described above, or a pharmaceutical composition as described above.

The invention is further illustrated, but not limited by the following Examples in which the following general procedures were used unless stated otherwise.

N,N-Dimethylformamide (DMF) was dried over 4 Å molecular sieves. Anhydrous tetrahydrofuran (THF) was obtained from Aldrich SURESEAL™ bottles. Other commercially available reagents and solvents were used without further purification unless otherwise stated. Organic solvent extracts were dried over anhydrous MgSO₄. ¹H, ¹³C and ¹⁹F NMR were recorded on Bruker WM200, WM250, WM300 or WM400 instruments using Me₂SO-d₆ with Me₄Si or CCl₃F as internal standard as appropriate, unless otherwise stated. Chemical shifts are in d (ppm) and peak multiplicities are designated as follows: s, singlet; d, doublet; dd, doublet of doublets; t, triplet; dt, doublet of triplets; q, quartet; m, multiplet; br, broad. Mass spectra were recorded on VG 12-12 quadruple, VG 70-250 SE, VG ZAB 2-SE or a VG modified AEI/Kratos MS9 spectrometers. For TLC analysis, Merck precoated TLC plates (silica gel 60 F254, d=0.25 mm) were used. Flash chromatography was performed on silica (Merck Kieselgel: Art.9385). Melting point determinations were performed on a Kofler block or with a Büchi melting point apparatus and are uncorrected. All temperatures are in degrees Centigrade.

### EXAMPLE 1

Preparation of Compound No. 1 in Table 2

Route A

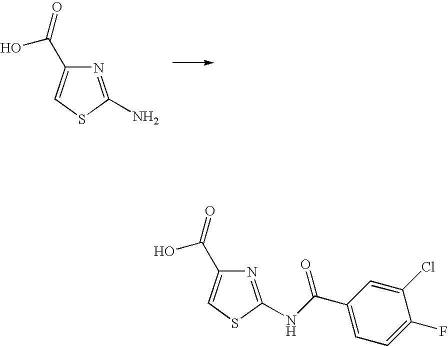

### 2-[(3-Chloro-4-fluorobenzoyl)amino]-1,3-thiazole-4-carboxylic acid

To a solution of 2-amino-4-thiazole carboxylic acid hydrobromide (2.24 g) in acetonitrile (15 ml) was added 3-chloro-4-fluorobenzoyl chloride (1.92 g) and diisopropylethylamine (3.6 ml). The resulting solution was stirred at room temperature for 24 hours before being poured into water (30 ml). The mixture was acidified to pH 1 with conc. HCl and the resulting solid 2-[(3-chloro-4-fluorobenzoyl)amino]-1,3-thiazole-4-carboxylic acid was filtered, washed with water and dried in vacuo at 50° C. (2.28 g).

LCMS M/z(+) 301 (M+H⁺)

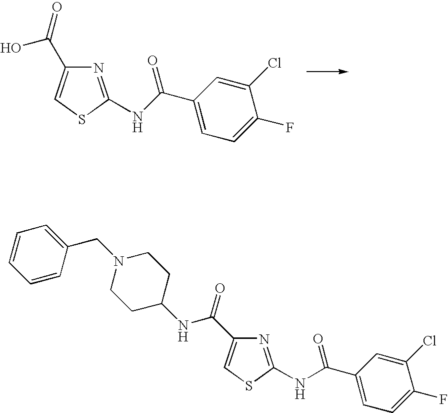

### N-(1-Benzylpiperidin-4-yl)-2-[(3-chloro-4-fluorobenzoyl)amino]-1,3-thiazole-4-carboxamide

HATU (0.38 g) was added to a solution of 2-[(3-chloro-4-fluorobenzoyl)amino]-1,3-thiazole-4-carboxylic acid (0.3 g) and diisopropylethylamine (0.16 ml) in N,N-dimethylformamide (4 ml) at ambient temperature. After 15 minutes 1-benzyl-4-aminopiperidine (0.2 g) was added and the mixture stirred at room temperature overnight. The reaction mixture was partitioned between ethyl acetate (10 ml) and aqueous potassium carbonate (5 ml). The organic layer was separated, dried over sodium sulfate and evaporated. The residue was purified using reverse phase HPLC eluting with a mixture of 5-95% acetonitrile in water to yield N-(1-benzylpiperidin-4-yl)-2-[(3-chloro-4-fluorobenzoyl)amino]-1,3-thiazole-4-carboxamide as a white solid (42 mg).

LCMS M/z(+) 473 (M+H⁺)

¹H-NMR (CDCl₃): 2.05 (4H, m), 3.1 (2H, m), 3.35 (2H, d), 4.05 (1H, m), 4.27 (2H, d), 7.46 (3H, m), 7.63 (1H, t), 7.67 (2H, m), 7.76 (1H, d), 7.93 (1H, s), 8.14 (1H, m), 8.37 (1H, q)

Alternative bis-amines to 1-benzyl-4-aminopiperidine can be used in this sequence to produce compounds with different basic side chains.

### EXAMPLE 2

Preparation of Compound No. 21 in Table 2

Route B

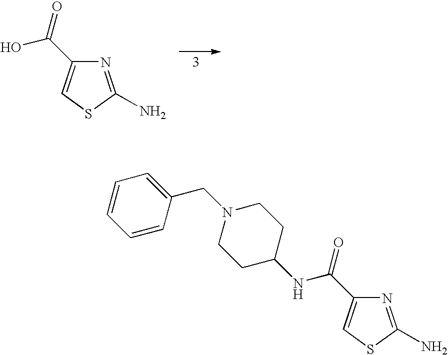

### 2-Amino-N-(1-benzylpiperidin-4-yl)-1,3-thiazole-4-carboxamide

HATU (3.8 g) was added to a solution of 2-amino-4-thiazole carboxylic acid hydrobromide (2.24 g) and diisopropylethylamine (4.0 ml) in N,N-dimethylformamide (20 ml) at ambient temperature. After 5 minutes 1-benzyl-4-aminopiperidine (1.9 g) was added and the mixture stirred at room temperature for 18 hours. The reaction mixture was partitioned between ethyl acetate (30 ml) and aqueous potassium carbonate (15 ml). The organic layer was separated, dried over sodium sulfate and evaporated to a brown residue of 2-amino-N-(1-benzylpiperidin-4-yl)-1,3-thiazole-4-carboxamide which solidified (2.46 g).

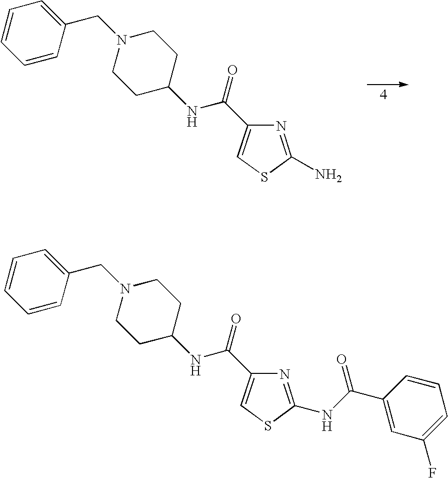

### N-(1-Benzylpiperidin-4-yl)-2-[(3-fluorobenzoyl)amino]-1,3-thiazole-4-carboxamide

Diisopropylethylamine (0.16 ml) was added to a stirred mixture of 3-fluorobenzoyl chloride (0.08 g) and 2-amino-N-(1-benzylpiperidin-4-yl)-1,3-thiazole-4-carboxamide (0.15 g) in acetonitrile (4 ml). Stirring was continued at room temperature for 18 hours before the mixture was partitioned between ethyl acetate (10 ml) and aqueous potassium carbonate (5 ml). The organic layer was separated, dried over sodium sulfate and evaporated. The residue was purified using reverse phase HPLC eluting with a gradient of 5-95% acetonitrile in water to produce N-(1-benzylpiperidin-4-yl)-2-[(3-fluorobenzoyl)amino]-1,3-thiazole-4-carboxamide as a white solid (104 mg).

LCMS M/z(+) 439 (M+H⁺)

Alternative acid chlorides to 3-fluorobenzoyl chloride can be used in this sequence to produce compounds with different side chains.

### EXAMPLE 3

Alternative Preparation of Compound No. 1 in Table 2

Route C

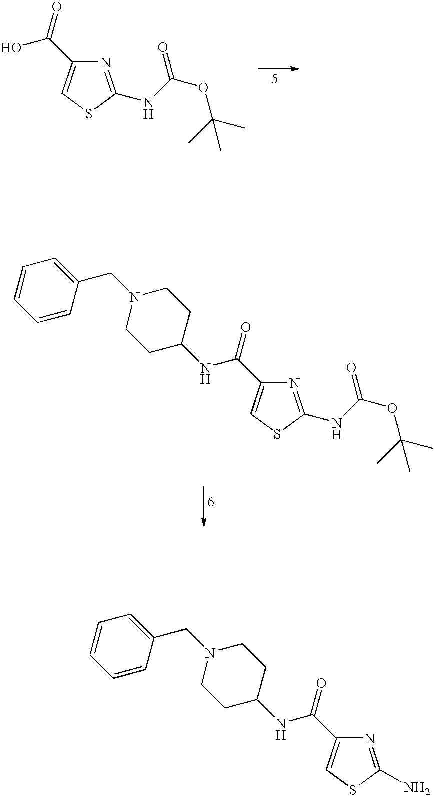

### 2-Amino-N-(1-benzylpiperidin-4-yl)-1,3-thiazole-4-carboxamide

HATU (0.95 g) was added to a stirred solution of 2-Boc-amino-4-thiazole carboxylic acid (0.6 g) and diisopropylethylamine (0.4 ml) in N,N-dimethylformamide (6 ml) at room temperature. After 15 minutes, 1-benzyl-4-aminopiperidine (0.48 g) was added and the whole stirred at room temperature overnight. The reaction mixture was partitioned between water (20 ml) and ethyl acetate (3×10 ml). The organic layer was dried over sodium sulfate and evaporated. The residue was re-dissolved in methanol and applied to a pre-wetted SCX column, which was the washed with methanol before eluting with 2M ammonia in methanol. The eluant was evaporated to an oily solid, which was dissolved in dichloromethane (10 ml) and treated with trifluoroacetic acid (3 ml) at room temperature overnight. Water (50 ml) followed by solid potassium carbonate was added to neutralize any remaining trifluoroacetic acid. The organic layer was separated, evaporated and re-dissolved in ethyl acetate (15 ml) before excess hydrochloric acid (2M in diethyl ether) solution was added. The white solid given was collected by evaporation, washed with ethyl acetate and dried under vacuum at room temperature to give 2-amino-N-(1-benzylpiperidin-4-yl)-1,3-thiazole-4-carboxamide (0.65 g).

LCMS M/z(+) 317 (M+H⁺)

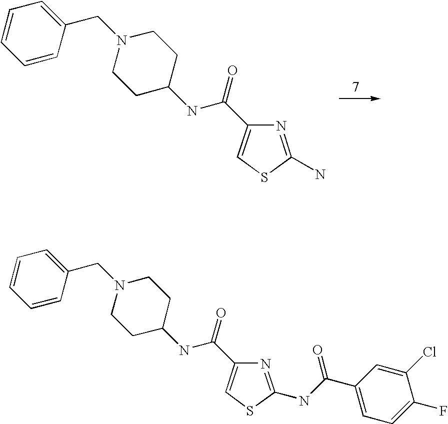

### N-(1-Benzylpiperidin-4-yl)-2-[(3-chloro-4-fluorobenzoyl)amino]-1,3-thiazole-4-carboxamide

3-chloro-4-fluorobenzoyl chloride (0.38 g) was added to a mixture of 2-amino-N-(1-benzylpiperidin-4-yl)-1,3-thiazole-4-carboxamide (0.32 g) and diisopropylethylamine (0.32 ml) in dioxane (5 ml) at ambient temperature before heating at 60° C. for 30 minutes. The mixture was cooled and partitioned between ethyl acetate (15 ml) and aqueous potassium carbonate (5 ml). The organic layer was separated, dried over sodium sulfate and evaporated. The residue was purified by silica gel column chromatography eluting with a gradient from 0-20% methanol in dichloromethane to produce N-(1-benzylpiperidin-4-yl)-2-[(3-chloro-4-fluorobenzoyl)amino]-1,3-thiazole-4-carboxamide as a white solid (310 mg).

¹H-NMR (CDCl₃): 2.05 (4H, m), 3.1 (2H, m), 3.35 (2H, d), 4.05 (1H, m), 4.27 (2H, d), 7.46 (3H, m), 7.63 (1H, t), 7.67 (2H, m), 7.76 (1H, d), 7.93 (1H, s), 8.14 (1H, m), 8.37 (1H, q); LCMS M/z(+) 473 (M+H⁺).

Alternative acid chlorides to 3-chloro-4-fluorobenzoyl chloride can be used in this sequence to produce compounds with different side chains.

### EXAMPLE 4

Preparation of Compound No. 24 in Table 2

Route D

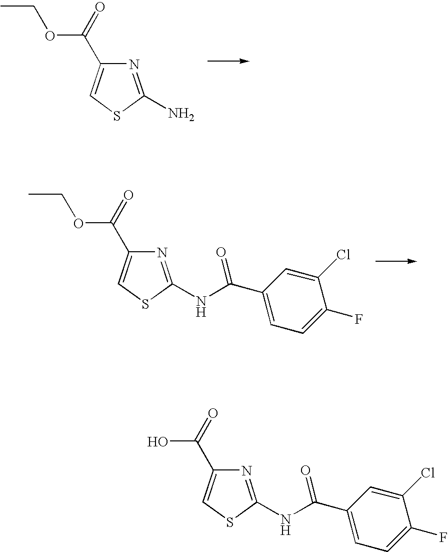

### 2-[(3-Chloro-4-fluorobenzoyl)amino]-1,3-thiazole-4-carboxylic acid

Solid 3-chloro-4-fluorobenzoyl chloride (0.9 g) was added to a stirred solution of ethyl 2-aminothiazole-4-carboxylate (1.0 g) and diisopropylethylamine (1.0 ml) in tetrahydrofuran (10 ml) at ambient temperature. Stirring was continued overnight. The solvent was removed under vacuum and the residue partitioned between ethyl acetate (10 ml) and dilute aqueous potassium carbonate (10 ml). The organic layer was dried with sodium sulfate and evaporated.

The residue was stirred in a mixture of methanol before lithium hydroxide monohydrate (0.63 g) was added. After stirring overnight, the clear solution was acidified to pH 1 using conc. hydrochloric acid. The precipitated acid was collected on a filter, washed with water and dried under vacuum at 60° C. to give solid 2-[(3-chloro-4-fluorobenzoyl)amino]-1,3-thiazole-4-carboxylic acid (1.23 g).

LCMS M/z(+) 301 (M+H⁺).

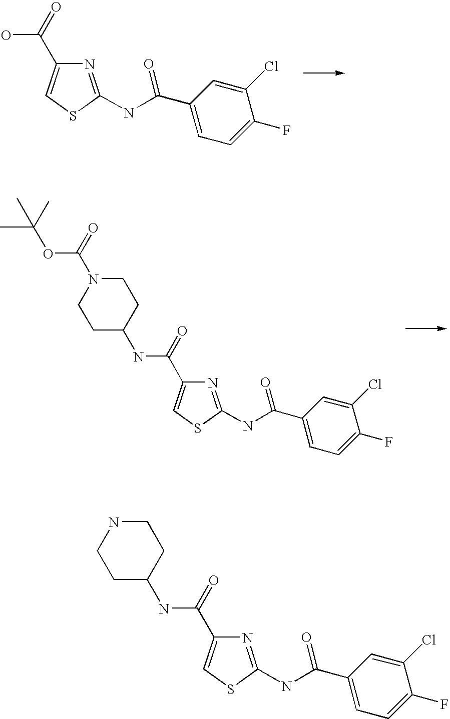

### 2-[(3-Chloro-4-fluorobenzoyl)amino]-N-piperidin-4-yl-1,3-thiazole-4-carboxamide

HATU (1.6 g; 4.26 mmol) was added to a solution of 2-[(3-chloro-4-fluorobenzoyl)amino]-1,3-thiazole-4-carboxylic acid (1.23 g) and diisopropylethylamine (0.7 ml) in N,N-dimethylformamide (10 ml). After stirring at room temperature for 10 minutes, 4-amino-N-Boc-piperidine (0.85 g) was added and the whole stirred at ambient temperature overnight.

The mixture was partitioned between ethyl acetate (15 ml) and dilute aqueous potassium carbonate solution (10 ml). The organic layer was separated, dried with sodium sulfate and evaporated. The residue was re-dissolved in dichloromethane (15 ml) and stirred with 4.0 M hydrochloric acid in dioxane (10 ml) at room temperature for 18 hours. The resulting slurry was diluted with dichloromethane (15 ml) and filtered. The residual solid was washed with dichloromethane and dried in vacuo at room temperature to give solid 2-[(3-chloro-4-fluorobenzoyl)amino]-N-piperidin-4-yl-1,3-thiazole-4-carboxamide, (1.45 g)

LCMS M/z(+) 383 (M+H⁺).

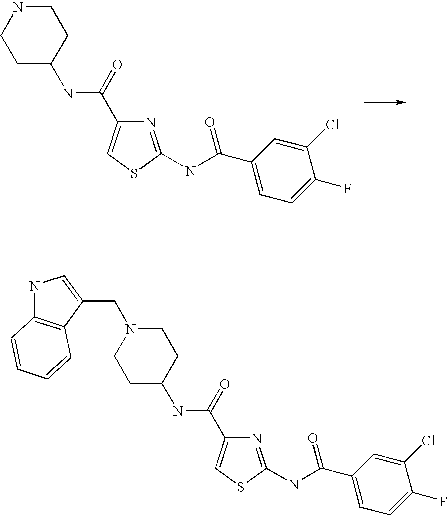

### 2-[(3-chloro-4-fluorobenzoyl)amino]-N-[1-(1H-indol-3-ylmethyl)piperidin-4-yl]-1,3-thiazole-4-carboxamide

Solid 2-[(3-chloro-4-fluorobenzoyl)amino]-N-piperidin-4-yl-1,3-thiazole-4-carboxamide HCl salt (0.12 g) followed by diisopropylethylamine (0.16 ml) was added to a solution of indole-3-carboxyaldehyde (1 mmol) in tetrahydrofuran (2 ml) and acetonitrile (2 ml). Sodium triacetoxyborohydride (0.22 g) was then added and the resulting mixture stirred at ambient temperature for 18 hours. The reaction mixture was then partitioned between water (5 ml) and ethyl acetate (10 ml). The organic layer was separated, dried over sodium sulphate and evaporated. Purification was by HPLC eluting with a gradient of 5-95% acetonitrile in water, which produced 2-[(3-chloro-4-fluorobenzoyl)amino]-N-[1-(1H-indol-3-ylmethyl)piperidin-4-yl]-1,3-thiazole-4-carboxamide as a white solid (97 mg).

LCMS M/z(+) 512 (M+H⁺).

Alternative aldehydes to indole-3-carboxyaldehyde can be used in this sequence to produce compounds with different basic side chains.

### EXAMPLE 5

Preparation of Compound No. 26 in Table 2

Route E

### N-(1-benzylpiperidin-4-yl)-5-nitrothiophene-3-carboxamide

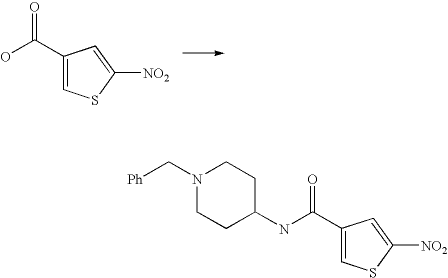

1-Chloro-N,N-2-dimethyl-1-propenylamine (2.53 ml) was added to a stirred solution of 2-nitrothiophene-4-carboxylic acid (3.0 g) in dichloromethane (30 ml). After 30 mins diisopropylethylamine (5.95 ml) was added, followed by dropwise addition of 4-amino-1-benzyl-piperidine (3.61 g) and stirring continued for 18 hours. Water (20 ml) was added, the layers separated and the organics concentrated in vacuo. The residue was subjected to silica gel column chromatography eluting with 0-10% methanol/dichloromethane. The product fractions were concentrated in vacuo, and triturated with ether to yield N-(1-benzylpiperidin-4-yl)-5-nitrothiophene-3-carboxamide as a pale brown solid (3.63 g).

¹H-NMR (D₆-DMSO): 1.5 (2H, m), 1.8 (2H, d), 2.0 (2H, t), 2.8 (2H, d), 3.5 (2H, s), 3.7 (1H, m), 7.3 (5H, m), 8.3 (1H, d), 8.5 (1H, s), 8.55 (1H, s). LCMS M/z(+) 346 (MH⁺)

### 5-Amino-N-(1-benzylpiperidin-4-yl)thiophene-3-carboxamide

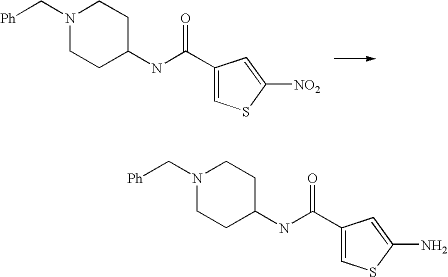

Iron powder (900 mg) and ammonium chloride (86 mg) were added to a solution of N-(1-benzylpiperidin-4-yl)-5-nitrothiophene-3-carboxamide (800 mg) in ethanol/water (2:1, 15 ml) and the mixture heated to 70° C. for 17 hours. The mixture was then cooled to room temperature, celite added and stirring continued for 10 minutes. This suspension was then filtered through a pad of celite, and the filtrate concentrated in vacuo. The residue was subjected to SCX2 chromatography eluting initially with methanol/dichloromethane (0-20%) followed by 0-10% (2M ammonia in methanol/dichloromethane) to elute the product. Product fractions were concentrated in vacuo, adsorbed onto silica and subjected to column chromatography eluting with 0-10% methanol/dichloromethane. Product fractions were concentrated in vacuo to yield 5-amino-N-(1-benzylpiperidin-4-yl)thiophene-3-carboxamide as a brown solid (220 mg).

LCMS M/z(+) 316 (MH⁺).

### N-(1-Benzylpiperidin-4-yl)-5-[(3-chloro-4-fluorobenzoyl)amino]thiophene-3-carboxamide

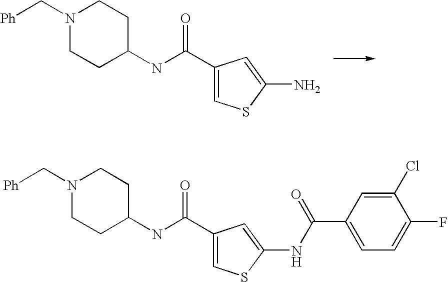

3-Chloro-4-fluoro benzoyl chloride (67 mg) was added to a solution of 5-amino-N-(1-benzylpiperidin-4-yl)thiophene-3-carboxamide (100 mg) in dichloromethane (5 ml) and stirred at room temperature for 30 minutes. The mixture was concentrated in vacuo, adsorbed onto silica and purified by silica gel chromatography, eluting with 0-20% methanol/dichloromethane. Trituration with ether yielded N-(1-benzylpiperidin-4-yl)-5-[(3-chloro-4-fluorobenzoyl)amino]thiophene-3-carboxamide as a brown solid (120 mg).

¹H-NMR (D₆-DMSO): 1.6 (2H, m), 1.8 (2H, m), 2.0 (2H, m), 2.8 (2H, m), 3.5 (2H, s), 3.7 (1H, m), 7.25 (1H, s), 7.4 (5H, m), 7.65 (2H, m), 8.65 (2H, m), 8.25 (1H, dd), 11.7 (1H, s). LCMS M/z(+) 472/474 (MH⁺)

Alternative acid chlorides to 3-chloro-4-fluorobenzoyl chloride can be used in this sequence to produce compounds with different side chains.

### EXAMPLE 6

Preparation of Compound No. 27 in Table 2

Route F

### tert-butyl 4-{[(2-amino-1,3-thiazol-4-yl)carbonyl]amino}piperidine-1-carboxylate

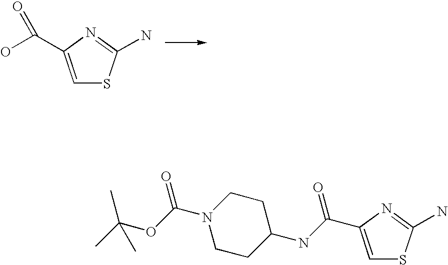

2-Amino-4-thiazolecarboxylic acid hydrobromide (2.67 g) and 1-BOC-4-aminopiperidine hydrochloride (2.81 g) were suspended in dichloromethane (100 ml) and stirred at room temperature. Dimethylaminopyridine (5.8 g) was added followed by 1-(3-dimethylaminopropyl)-3-ethylcarbodiimide hydrochloride (2.5 g). To assist with solubility, N,N-dimethylformamide (5 ml) was added and stirring was continued overnight. The reaction mixture was concentrated in vacuo and the residue partitioned between ethyl acetate (100 ml) and brine (50 ml). The organic portion was concentrated in vacuo and adsorbed onto silica and purified by silica gel chromatography, eluting with 0-5% methanol/dichloromethane to yield tert-butyl 4-{[(2-amino-1,3-thiazol-4-yl)carbonyl]amino}piperidine-1-carboxylate as a brown brittle foam (1.7 g).

¹H-NMR (D₆-DMSO): 1.4 (11H, m), 1.7 (2H, m), 2.8 (2H, m), 3.9 (3H, m), 7.0 (2H, s), 7.15 (1H, s), 7.5 (1H,d). LCMS M/z(+) 327 (MH⁺)

### tert-butyl 4-[({2-[(4-fluorobenzoyl)amino]-1,3-thiazol-4-yl}carbonyl)amino]-piperidine-1 carboxylate

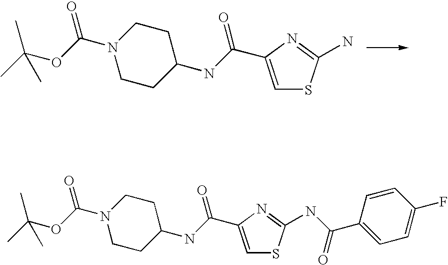

tert-butyl 4-{[(2-amino-1,3-thiazol-4-yl)carbonyl]amino}piperidine-1-carboxylate (300 mg), triethylamine (0.39), 4-fluorobenzoyl chloride (0.22 ml) and tetrahydrofuran (6 ml) were sealed in a microwave vessel and microwaved for 2 hours at 150° C. The reaction mixture was concentrated in vacuo and the residue partitioned between ethyl acetate (25 ml) and brine (15 ml). The organic portion was concentrated in vacuo and adsorbed onto silica and purified by silica gel chromatography, eluting with 0-10% methanol/dichloromethane to yield tert-butyl 4-[({2-[(4-fluorobenzoyl)amino]-1,3-thiazol-4-yl}carbonyl)amino]-piperidine-1 carboxylate as a brown gum (340 mg).

LCMS M/z(−) 447 (MH⁻)

### 2-[(4-fluorobenzoyl)amino]-N-piperidin-4-yl-1,3-thiazole-4-carboxamide hydrochloride

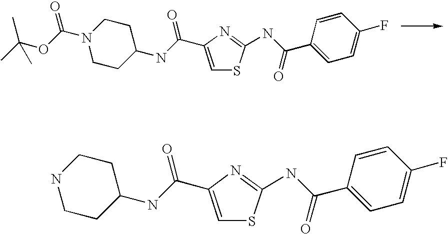

tert-butyl 4-[({2-[(4-fluorobenzoyl)amino]-1,3-thiazol-4-yl}carbonyl)amino]-piperidine-1 carboxylate (340 mg) was dissolved in methanol (10 ml) and 4N hydrogen chloride in 1,4-dioxan (5 ml) and stirred at room temperature overnight. The mixture was concentrated in vacuo and azeotroped once with toluene to yield 2-[(4-fluorobenzoyl)amino]-N-piperidin-4-yl-1,3-thiazole-4-carboxamide hydrochloride as a pale yellow solid (330 mg).

LCMS M/z(+) 349 (MH⁺)

### 2-[(4-fluorobenzoyl)amino]-N-[1-(1H-indol-3-ylmethyl)piperidin-4-yl]-1,3-thiazole-4-carboxamide

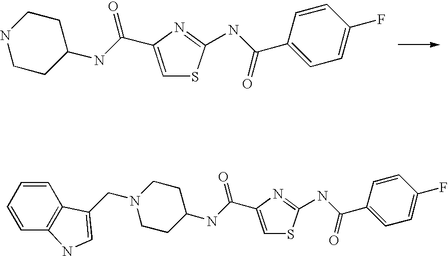

2-[(4-fluorobenzoyl)amino]-N-piperidin-4-yl-1,3-thiazole-4-carboxamide hydrochloride (115 mg) was stirred at room temperature in tetrahydrofuran (12 ml) as a suspension. N,N-diisopropylethylamine (0.21 ml) was added followed by indole-3-carboxyaldehyde (87 mg) and magnesium sulphate (30 mg). After 5 minutes, sodium triacetoxyborohydride (127 mg) was added. Stirring was continued overnight and the reaction mixture partitioned between ethyl acetate (25 ml) and brine (10 ml). The organic portion was concentrated in vacuo and adsorbed onto silica and purified by silica gel chromatography, eluting with 0-12.5% methanol/dichloromethane to yield 2-[(4-fluorobenzoyl)amino]-N-[1-(1H-indol-3-ylmethyl)piperidin-4-yl]-1,3-thiazole-4-carboxamide as a light brown solid (60 mg).

¹H-NMR (D₆-DMSO): 1.9 (4H, m), 3.3 (6H, m), 4.4 (1H, m), 7.1 (2H, m), 7.4 (3H, m), 7.6 (1H, s), 7.8 (2H, d), 7.9 (1H, s), 8.2 (2H, m), 11.4 (1H, s), 12.8 (1H, s). LCMS M/z(+) 478 (MH⁺)

Preparation of Compound No. 28 in Table 2

### N-[1-(1H-indol-3-ylmethyl)piperidin-4-yl]-2-(2-naphthoylamino)-1,3-thiazole-4-carboxamide

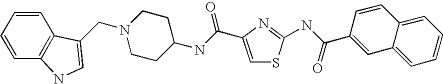

N-[1-(1H-indol-3-ylmethyl)piperidin-4-yl]-2-(2-naphthoylamino)-1,3-thiazole-4-carboxamide was prepared via route F using 2-Naphthoyl chloride instead of 4-fluorobenzoyl chloride as a pale yellow glass (100 mg).

¹H-NMR (D₆-DMSO): 1.9 (4H, m), 3.0 (5H, m), 4.0 (2H, m), 7.1 (2H, m), 7.45 (1H, d), 7.6 (3H, m), 7.8 (2H, m), 7.95 (1H, s), 8.1 (4H, m), 8.8 (1H, s). LCMS M/z(+) 510 (MH⁺)₋

Preparation of Compound No. 29 in Table 2

### 2-[(1,3-benzodioxol-5-ylcarbonyl)amino]-N-[1-(1H-indol-3-ylmethyl)piperidin-4-yl]-1,3-thiazole-4-carboxamide

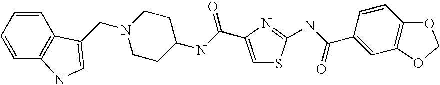

2-[(1,3-benzodioxol-5-ylcarbonyl)amino]-N-[1-(1H-indol-3-ylmethyl)piperidin-4-yl]-1,3-thiazole-4-carboxamide was prepared via route F using piperonyloyl chloride instead of 4-fluorobenzoyl chloride as a white solid (43 mg).

¹H-NMR (D₆-DMSO): 1.8 (4H, m), 3.4 (7H, m), 6.1 (1H, s), 7.1 (3H, m), 7.4 (1H, d), 7.5 (1H, m), 7.65 (1H, s), 7.7 (3H, m), 7.85 (1H, s). LCMS M/z(+) 504 (MH⁺)

Preparation of Compound No. 30 in Table 2

### N-[1-(1H-indol-3-ylmethyl)piperidin-4-yl]-2-[(3-thienylcarbonyl)amino]-1,3-thiazole-4-carboxamide

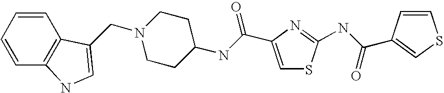

N-[1-(1H-indol-3-ylmethyl)piperidin-4-yl]-2-[(3-thienylcarbonyl)amino]-1,3-thiazole-4-carboxamide was prepared via route F using thiophene-3-carbonyl chloride instead of 4-fluorobenzoyl chloride as a light brown solid (23 mg).

¹H-NMR (D₆-DMSO): 1.9 (4H, m), 2.6 (4H, m), 3.5 (2H, m), 3.9 (1H, m), 7.1 (2H, m), 7.4 (2H, d), 7.7 (4H, m), 7.8 (1H, s), 8.6 (1H,s). LCMS M/z(+) 466 (MH⁺)

### EXAMPLE 7

Biological Assays

a) MCP-1 Mediated Calcium Flux in THP-1 Cells

The human monocytic cell line THP-1 was grown in a synthetic cell culture medium RPMI 1640 supplemented with 10% foetal calf serum, 6 mM glutainine and Penicillin-Streptomycin (at 50 IU/ml penicillin, 50 μg streptomycin/ml, Gibco BRL). THP-1 cells were washed in assay buffer comprising of HBSS with Ca²⁺ and Mg²⁺ (without phenol red) (Gibco BRL)+20 mM HEPES+0.71 mg/ml Propenecid+2 mls/litre CaCl₂ 1M (BDH)+0.3 mg/ml BSA (Sigma) pH 7.4 and resuspended in the same buffer at a density of 1×10⁶ cells/ml. The cells were then loaded with assay buffer+1 mM FLUO-4 (molecular probes) for 40 min at 37° C., washed twice in assay buffer, and resuspended at 2×10⁵ cells/ml. 100 μl of the cell suspension was added to the wells of black clear-bottomed 96 well plates, to give 2×10⁴ cells/well. Cells were pelleted by centrifugation and washed with assay buffer. 100 ul of buffer+50 ul of compound was added to wells and incubated for 20 mins at (37° C.). Fluorescence was recorded using a FLIPR (FLuorometric Imaging Plate Reader—Molecular Devices). Cells were stimulated by addition of hMCP-1 to the wells.

Stimulation of THP-1 cells with hMCP-1 induced a rapid, transient rise in [Ca²⁺]i in a specific and dose dependent manner. Dose response curves indicated an approximate EC₅₀ of 4 nm. Compounds were dissolved in DMSO (10 mM) and were assayed for inhibition of calcium release over concentration ranges starting at 10 μM.

Certain compounds described above were tested in this screen and found to be active. For example, compound No. 13 in Table 2 had an IC50 of 0.597 μM and compound No. 22 in Table 2 had an IC50 of 0.314 μM.

b) hMCP-1 Receptor-Binding Assay

i) Cloning and Expression of hMCP-1 Receptor

The MCP-1 receptor B (CCR2B) cDNA was cloned by PCR from THP-1 cell RNA using suitable oligonucleotide primers based on the published MCP-1 receptor sequences (Charo et al., 1994, Proc. Natl. Acad. Sci. USA, 91, 2752). The resulting PCR products were cloned into vector PCR-II™ (InVitrogen, San Diego, Calif.). Error free CCR2B cDNA was subcloned as a Hind III-Not I fragment into the eukaryotic expression vector pCDNA3 (InVitrogen) to generate pCDNA3/CC—CKR2A and pCDNA3/CCR2B respectively.

Linearized pCDNA3/CCR2B DNA was transfected into CHO—K1 cells by calcium phosphate precipitation (Wigler et al., 1979, Cell, 16, 777). Transfected cells were selected by the addition of Geneticin Sulphate (G418, Gibco BRL) at 1 mg/ml, 24 hours after the cells had been transfected. Preparation of RNA and Northern blotting were carried out as described previously (Needham et al., 1995, Prot. Express. Purific., 6, 134). CHO—K1 clone 7 (CHO—CCR2B) was identified as the highest MCP-1 receptor B expressor.

ii) Preparation of Membrane Fragments

CHO—CCR2B cells were grown in DMEM supplemented with 10% foetal calf serum, 2 mM glutamine, 1× Non-Essential Amino Acids, 1× Hypoxanthine and Thymidine Supplement and Penicillin-Streptomycin (at 50 μg streptomycin/ml, Gibco BRL). Membrane fragments were prepared using cell lysis/differential centrifugation methods as described previously (Siciliano et al., 1990, J. Biol. Chem., 265, 19658). Protein concentration was estimated by BCA protein assay (Pierce, Rockford, Ill.) according to the manufacturer's instructions.

iii) Assay

¹²⁵I-labeled MCP-1 was prepared using Bolton and Hunter conjugation (Bolton et al., 1973, Biochem. J., 133, 529; Amersham International plc].

Test compounds were dissolved in DMSO and further diluted in assay buffer (50 mM HEPES, 1 mM CaCl₂, 5 nM MgCl₂, 0.03% BSA, pH 7.2) to give a range of concentrations starting with a top final concentration of 10 uM. All incubations had a 100 ul final volume and a DMSO concentration of 1%. Incubations contained 200 pM ¹²⁵I-labeled MCP-1 (Amersham Pharmacia), 2.5 mg/ml Scintillation proximity assay beads (Amersham Pharmacia RPNQ) and approx 5 ug CHO—CCR2B cell membranes. Non-specific binding was determined by the inclusion of a 1 uM unlabeled MCP-1 in the place of test compound. Total binding was determined in the presence of 1% DMSO without compound. Incubations were performed in sealed optiplates and kept at room temperature for 16 hours after which the plates were counted on a Packard TopCount (Packard TopCount™). Dose-response curves were generated from duplicate date points and IC₅₀ values were calculated using GraphPad Prizm® software. Percent inhibitions were calculated for single concentrations of compound by using the following formula 100−((compound binding minus non-specific binding)/(total binding minus non-specific binding)×100).

In the above assay each compound set out in the Examples below showed an IC₅₀ value of better than 20 μmol

### EXAMPLE 8

Pharmaceutical Compositions

This Example illustrates, but is not intended to limit, representative pharmaceutical dosage forms of the invention as defined herein (the active ingredient being termed “Compound X”), for therapeutic or prophylactic use in humans:

### EXAMPLE A

(a)

| Tablet I | mg/tablet |
|---|---|
| Compound X. | 100 |
| Lactose Ph.Eur | 182.75 |
| Croscarmellose sodium | 12.0 |
| Maize starch paste (5% w/v paste) | 2.25 |
| Magnesium stearate | 3.0 |

(b)

| Tablet II | mg/tablet |
|---|---|
| Compound X | 50 |
| Lactose Ph.Eur | 223.75 |
| Croscarmellose sodium | 6.0 |
| Maize starch | 15.0 |
| Polyvinylpyrrolidone (5% w/v paste) | 2.25 |
| Magnesium stearate | 3.0 |

(c)

| Tablet III | mg/tablet |
|---|---|
| Compound X | 1.0 |
| Lactose Ph.Eur | 93.25 |
| Croscarmellose sodium | 4.0 |
| Maize starch paste (5% w/v paste) | 0.75 |
| Magnesium stearate | 1.0 |

(d)

| Capsule | mg/capsule |
|---|---|
| Compound X | 10 |
| Lactose Ph.Eur | 488.5 |
| Magnesium | 1.5 |

(e)

| Injection I | (50 mg/ml) | |
|---|---|---|
| Compound X | 5.0% | w/v |
| 1M Sodium hydroxide solution | 15.0% | v/v |
| 0.1M Hydrochloric acid | to adjust pH to 7.6 | |
| Polyethylene glycol 400 | 4.5% | w/v |

| | |
|---|---|
| Water for injection | to 100% |

(f)

| Injection II | (10 mg/ml) | |
|---|---|---|
| Compound X | 1.0% | w/v |
| Sodium phosphate BP | 3.6% | w/v |
| 0.1M Sodium hydroxide solution | 15.0% | v/v |

| | |
|---|---|
| Water for injection | to 100% |

(g)

| | (1 mg/ml, | |
|---|---|---|
| Injection III | buffered to pH6) | |
| Compound X | 0.1% | w/v |
| Sodium phosphate BP | 2.26% | w/v |
| Citric acid | 0.38% | w/v |
| Polyethylene glycol 400 | 3.5% | w/v |

| | |
|---|---|
| Water for injection | to 100% |

(h)

| Aerosol I | mg/ml |
|---|---|
| Compound X | 10.0 |
| Sorbitan trioleate | 13.5 |
| Trichlorofluoromethane | 910.0 |
| Dichlorodifluoromethane | 490.0 |

(i)

| Aerosol II | mg/ml |
|---|---|
| Compound X | 0.2 |
| Sorbitan trioleate | 0.27 |
| Trichlorofluoromethane | 70.0 |
| Dichlorodifluoromethane | 280.0 |
| Dichlorotetrafluoroethane | 1094.0 |

(j)

| Aerosol III | mg/ml |
|---|---|
| Compound X | 2.5 |
| Sorbitan trioleate | 3.38 |
| Trichlorofluoromethane | 67.5 |
| Dichlorodifluoromethane | 1086.0 |
| Dichlorotetrafluoroethane | 191.6 |

(k)

| Aerosol IV | mg/ml |
|---|---|
| Compound X | 2.5 |
| Soya lecithin | 2.7 |
| Trichlorofluoromethane | 67.5 |
| Dichlorodifluoromethane | 1086.0 |
| Dichlorotetrafluoroethane | 191.6 |

(l)

| Ointment | ml | |
|---|---|---|
| Compound X | 40 | mg |
| Ethanol | 300 | μl |
| Water | 300 | μl |
| 1-Dodecylazacycloheptan-2-one | 50 | μl |
| Propylene glycol | to 1 | ml |

Note:
Compound X in the above formulations may comprise a compound as illustrated in herein.

The above formulations may be obtained by conventional procedures well known in the pharmaceutical art. The tablets (a)-(c) may be enteric coated by conventional means, for example to provide a coating of cellulose acetate phthalate. The aerosol formulations (h)-(k) may be used in conjunction with standard, metered dose aerosol dispensers, and the suspending agents sorbitan trioleate and soya lecithin may be replaced by an alternative suspending agent such as sorbitan monooleate, sorbitan sesquioleate, polysorbate 80, polyglycerol oleate or oleic acid.

1. The use of a compound of formula (I) 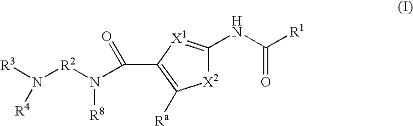
or a pharmaceutically acceptable salt or solvate thereof,
wherein
X¹ is nitrogen or CH,
X² is sulphur or NH,
R¹ is an optionally substituted alkyl, optionally substituted alkoxy, optionally substituted cycloalkyl, optionally substituted heterocyclyl or optionally substituted aryl ring, wherein two substituents may be joined together to form an optionally substituted fused bicyclic ring, which may contain hetero atoms,
R^{a} is hydrogen, C₁₋₃alkyl, C₂₋₄alkenyl, C₂₋₄alkynyl, trifluoromethyl, halo, amino, C₁₋₃alkylamino, di-C₁₋₃alkylamino, C₁₋₄alkoxy, hydroxy, thioC₁₋₄alkyl, or cyclopropyl;
R⁸ is hydrogen or an optionally substituted C₁₋₄alkyl group,
R² is an optionally substituted C₂₋₁₀straight or branched alkylene group, which is optionally interposed with a group NR^{b} where R^{b} is hydrogen or a C₁₋₃methyl group; or
R² together with R⁸ and the nitrogen atoms to which they are attached may form an optionally substituted cycloalkyl or heterocyclic ring,
R³ and R⁴ are independently selected from an optionally substituted C₁₋₁₀ alkyl group, an optionally substituted C₂₋₁₀ alkenyl group, an optionally substituted C₁₋₁₀ alkynyl group or an optionally substituted heterocyclic group,
or R³ and R⁴ together with the nitrogen atom to which they are attached form an optionally substituted heterocyclic ring, which optionally contains additional heteroatoms,
or R³ together with R² or R⁸ and the nitrogen atom(s) to which they are attached form an optionally substituted heterocyclic ring which optionally contains additional heteroatoms,
or R³ and R⁴ together with R² form an optionally substituted bridged ring structure, in the preparation of a medicament for the treatment of C—C chemokine mediated conditions.

2. The use according to claim 1 wherein, in the compound of formula (I), X¹ is nitrogen and X² is sulphur.

3. The use according to claim 1 or claim 2 wherein, in the compound of formula (I), R¹ is optionally substituted phenyl.

4. The use according to claim 3 wherein, in the compound of formula (I), R¹ is 4-fluoro-3-chloro-phenyl.

5. The use according to any one of claims 1 to 4 wherein, in the compound of formula (I), R¹ is pyridyl.

6. The use according to any one of the preceding claims wherein, in the compound of formula (I), R⁸ is hydrogen.

7. The use according to any one of claims 1 to 6 wherein, in the compound of formula (I), R⁴R³N— comprises a group of sub-formula (xx)-(xxv). 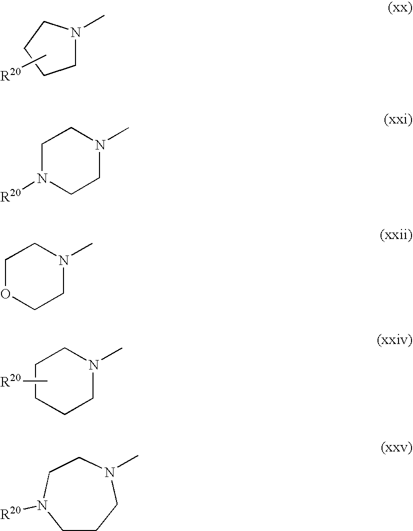
where R²⁰ is hydrogen or a substituent selected from alkyl, aralkyl such as benzyl, optionally substituted heterocyclic groups, and functional groups.

8. The use according to any one of claims 1 to 6 wherein, in the compound of formula (I), the group of sub-formula (x) 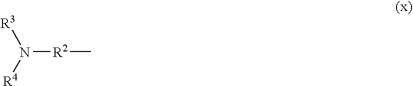
is a group of sub-formula (bb), (cc), (dd), (ee) or (ff) 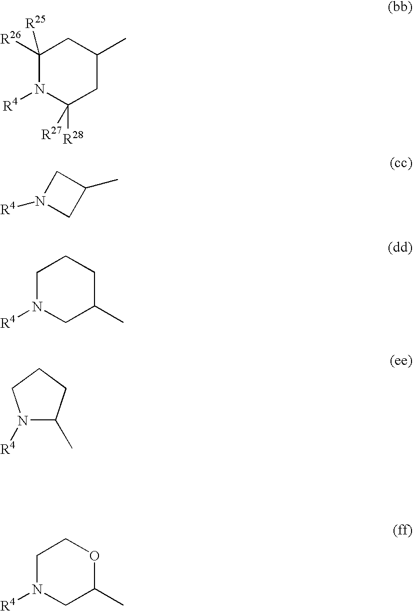
where R⁴ is as defined in claim 1, and R²⁵, R²⁶, R²⁷ and R²⁸ are independently selected from hydrogen or C₁₋₃alkyl.

9. The use according to claim 8 wherein the group of sub-formula (x) is a group of formula (bb) above.

10. A compound of formula (IA) 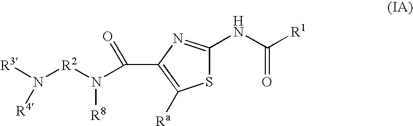
or a pharmaceutically acceptable salt or solvate thereof,
wherein R², R⁸, R^{a} and R¹ are as defined in claim 1, and where R^{3′} and R^{4′} are equivalent to R³ and R⁴ as defined in claim 1 respectively, provided that when R¹ is optionally substituted phenyl, and R^{a} and R⁸ is hydrogen, R^{3′} and R^{4′} are not both unsubstituted alkyl, or do not together with the nitrogen atom to which they are attached form a substituted piperazinyl ring; and further provided that when R¹ is an optionally substituted phenyl, R^{a} is hydrogen, C₁₋₃alkyl, or halo; R⁸ is hydrogen or a C₁₋₄alkyl group, and either R^{3′} and R² together with the nitrogen atom to which they are attached form a piperidinyl ring, or R^{3′} together R⁸ and the nitrogen atom(s) to which they are attached forms a piperazinyl ring, then R^{4′} is other than an unsubstituted C₁₋₆alkyl group; and yet further provided that when R¹ is an optionally substituted phenyl, R^{a} is hydrogen, C₁₋₃alkyl, or halo; and R² and R⁸ together with the nitrogen atom to which they are attached form a piperidinyl group, then R³′ and R^{4′} are not both unsubstituted C₁₋₆alkyl groups.

11. A compound according to claim 10 wherein R¹ is optionally substituted phenyl.

12. A compound according to claim 11 wherein R¹ is 4-fluoro-3-chloro-phenyl.

13. A compound according to any one of claims 10 to 12 wherein R¹ is pyridyl.

14. A compound according to claim any one claims 10 to 13 wherein R^{a} is hydrogen.

15. A compound according to any one of the claims 10 to 14 wherein R⁸ is hydrogen.

16. A compound according to any one of claims 10 to 15 wherein R⁴R³N— comprises a group of sub-formula (xx)-(xxv). 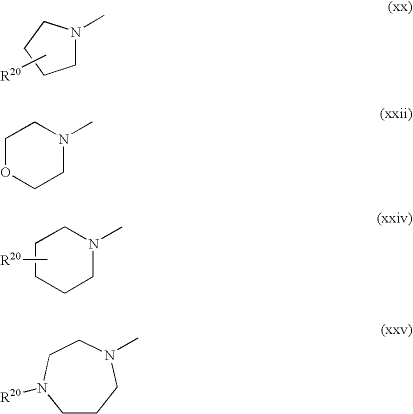
where R²⁰ is hydrogen or a substituent selected from alkyl, aralkyl such as benzyl, optionally substituted heterocyclic groups, and functional groups.

17. A compound according to any one of claims 10 to 15 wherein the group of sub-formula (x) 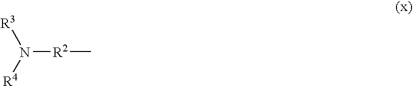
is a group of sub-formula (bb), (cc), (dd), (ee) or (ff) 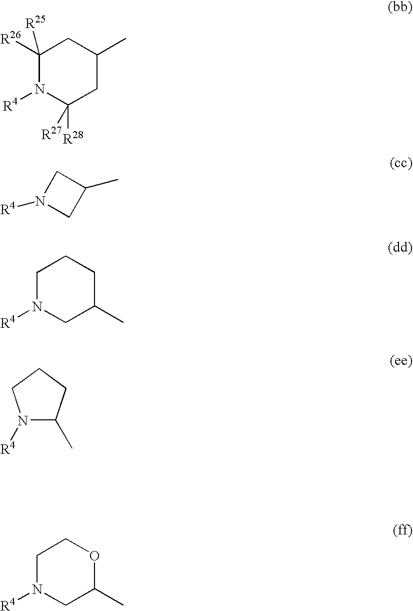
where R⁴ is as defined in claim 1, and R²⁵, R²⁶, R²⁷ and R²⁸ are independently selected from hydrogen or C₁₋₃alkyl.

18. A compound according to claim 17 wherein the group of sub-formula (x) is a group of formula (bb) above.

19. A compound according to claim 10 which is selected from:
tert-butyl (4-{[(1-benzylpiperidin-4-yl)amino]carbonyl}-1,3-thiazol-2-yl)carbamate,
N-(1-benzylpiperidin-4-yl)-2-[(3-chloro-4-fluorobenzoyl)amino]-1,3-thiazole-4-carboxamide,
2-[(3-chloro-4-fluorobenzoyl)amino]-N-(1-{[5-(hydroxymethyl)-2-furyl]methyl}piperidin-4-yl)-1,3-thiazole-4-carboxamide,
2-[(3-chloro-4-fluorobenzoyl)amino]-N-[1-(1H-imidazol-2-ylmethyl)piperidin-4-yl]-1,3-thiazole-4-carboxamide,
2-[(3-chloro-4-fluorobenzoyl)amino]-N-[1-(1H-imidazol-4-ylmethyl)piperidin-4-yl]-1,3-thiazole-4-carboxamide,
N-(1-benzylpiperidin-4-yl)-2-[(3,4-difluorobenzoyl)amino]-1,3-thiazole-4-carboxamide,
2-[(3-chloro-4-fluorobenzoyl)amino]-N-[1-(tetrahydro-2H-pyran-4-ylmethyl)piperidin-4-yl]-1,3-thiazole-4-carboxamide,
2-[(3-chloro-4-fluorobenzoyl)amino]-N-[1-(2-hydroxyethyl)piperidin-4-yl]-1,3-thiazole-4-carboxamide,
2-[(3-chloro-4-fluorobenzoyl)amino]-N-(1-{[6-(hydroxymethyl)pyridin-2-yl]methyl}piperidin-4-yl)-1,3-thiazole-4-carboxamide,
2-[(3-chloro-4-fluorobenzoyl)amino]-N-[1-(2,3-dihydro-1H-indol-3-ylmethyl)piperidin-4-yl]-1,3-thiazole-4-carboxamide,
2-[(3-chloro-4-fluorobenzoyl)amino]-N-[1-(1H-pyrazol-3-ylmethyl)piperidin-4-yl]-1,3-thiazole-4-carboxamide,
2-[(3-chloro-4-fluorobenzoyl)amino]-N-[1-(2-hydroxybenzyl)piperidin-4-yl]-1,3-thiazole-4-carboxamide,
2-[(3-chloro-4-fluorobenzoyl)amino]-N-{1-[(1-methyl-1H-pyrrol-2-yl)methyl]piperidin-4-yl}-1,3-thiazole-4-carboxamide,
2-[(3-chloro-4-fluorobenzoyl)amino]-N-{1-[4-(methylsulfonyl)benzyl]piperidin-4-yl}-1,3-thiazole-4-carboxamide,
2-[(3-chloro-4-fluorobenzoyl)amino]-N-[1-(1H-pyrrol-2-ylmethyl)piperidin-4-yl]-1,3-thiazole-4-carboxamide,
2-[(3-chloro-4-fluorobenzoyl)amino]-N-[1-(cyclopropylmethyl)piperidin-4-yl]-1,3-thiazole-4-carboxamide,
2-[(3-chloro-4-fluorobenzoyl)amino]-N-[1-(pyridin-3-ylmethyl)piperidin-4-yl]-1,3-thiazole-4-carboxamide,
N-(1-benzylpiperidin-4-yl)-2-[(3-chlorobenzoyl)amino]-1,3-thiazole-4-carboxamide,
N-(4-{[(1-benzylpiperidin-4-yl)amino]carbonyl}-1,3-thiazol-2-yl)isonicotinamide
N-(4-{[(1-benzylpiperidin-4-yl)amino]carbonyl}-1,3-thiazol-2-yl)pyridine-2-carboxamide,
N-(4-{[(1-benzylpiperidin-4-yl)amino]carbonyl}-1,3-thiazol-2-yl)nicotinamide,
N-(1-benzylpiperidin-4-yl)-2-[(3-fluorobenzoyl)amino]-1,3-thiazole-4-carboxamide,
N-(1-benzylpiperidin-4-yl)-2-[(3,4-dichlorobenzoyl)amino]-1,3-thiazole-4-carboxamide,
N-(1-benzylpiperidin-4-yl)-2-[(3-cyanobenzoyl)amino]-1,3-thiazole-4-carboxamide,
2-[(3-chloro-4-fluorobenzoyl)amino]-N-[1-(1H-indol-3-ylmethyl)piperidin-4-yl]-1,3-thiazole-4-carboxamide hydrochloride,
N-(1-benzylpiperidin-4-yl)-2-[(3-chloro-4-fluorobenzoyl)amino]-N-methyl-1,3-thiazole-4-carboxamide hydrochloride,
N-[(3S)-1-benzylpyrrolidin-3-yl]-2-[(3-chloro-4-fluorobenzoyl)amino]-1,3-thiazole-4-carboxamide hydrochloride,
3-chloro-4-fluoro-N-{4-[(4-pyrrolidin-1-ylpiperidin-1-yl)carbonyl]-1,3-thiazol-2-yl}benzamide,
2-[(3-chloro-4-fluorobenzoyl)amino]-N-(3-piperidin-1-ylpropyl)-1,3-thiazole-4-carboxamide, or
N-{4-[(4-benzyl-1,4-diazepan-1-yl)carbonyl]-1,3-thiazol-2-yl}-3-chloro-4-fluorobenzamide hydrochloride.
2-[(4-fluorobenzoyl)amino]-N-[1-(1H-indol-3-ylmethyl)piperidin-4-yl]-1,3-thiazole-4-carboxamide
N-[1-(1H-indol-3-ylmethyl)piperidin-4-yl]-2-(2-naphthoylamino)-1,3-thiazole-4-carboxamide
2-[(1,3-benzodioxol-5-ylcarbonyl)amino]-N-[1-(1H-indol-3-ylmethyl)piperidin-4-yl]-1,3-thiazole-4-carboxamide
N-[1-(1H-indol-3-ylmethyl)piperidin-4-yl]-2-[(3-thienylcarbonyl)amino]-1,3-thiazole-4-carboxamide

20. A process for preparing a compound of formula (IA) as defined in claim 10, which process comprises
(a) reacting a compound of formula (IV) 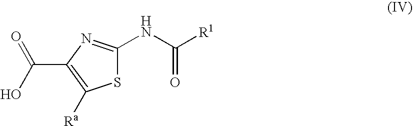
where R¹ and R^{a} are as defined in relation to formula (I), with a compound of formula (V) 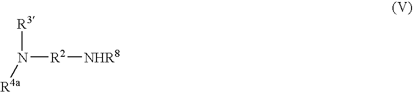
where R², R^{3′} and R⁸ are as defined in relation to formula (IA) and R^{4a} is a group R^{4′} as defined in claim 10, or a precursor thereof; or
(b) reacting a compound of formula (XIII) 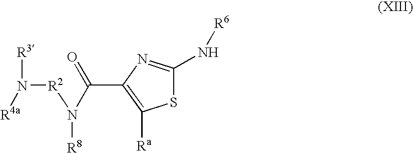
where R^{a}, R², R^{3′} and R⁸ are as defined in claim 10, R^{4a} is as defined in relation to formula (V), with a compound of formula (XIV) 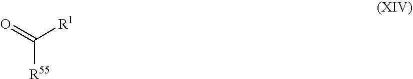
where R¹ are as defined in relation to formula (I) and R⁵⁵ is a leaving group: and thereafter if desired or necessary, converting any precursor groups R^{4a} to a group R^{4′} as defined in claim 10.

21. A compound of formula (I) as defined in claim 1 or a pharmaceutically acceptable salt or solvate thereof, for use in the treatment of C—C chemokine mediated disease.

22. A compound of formula (I) as defined in claim 1 for use in the treatment of CCR2B inflammatory disease.

23. A pharmaceutical composition comprising a compound according to any one of claims 10 to 19.

24. A method for inhibiting C—C chemokine mediated disease, which method comprises administering to a patient in need thereof, a compound of formula (I) as defined in any one of claims 1 to 9.

25. The use of according to any one of claims 1 to 9 for the preparation of a medicament for the treatment of CCR2B mediated inflammation.
